SEQUENCE LISTING (50 sequences)

SEQ ID NO: 1 - DNA, 850 bp, Homo sapiens
gcatagtgttaccatcaaccaccttaacttcatttttcttattcaatacctaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaacctaaaatgaaatgaaatcagacccttgtcttacaccataaacaaaaataaatttgaatgggttaaagaattaaactaagacctaaaaccataaaaatttttaaagaaatcaaaagaagaaaattctaatattcatgttgcagccgttttttgaatttgatatgagaagcaaaggcaacaaaaggaaaaataaagaagtgaggctacatcaaactaaaaaatttccacacaaaaaagaaaacaatgaacaaatgaaaggtgaaccatgaaatggcatatttgcaaaccaaatatttcttaaatattttggttaatatccaaaatatataagaaacacagatgattcaataacaaacaaaaaattaaaaataggaaaataaaaaaattaaaaagaagaaaatcctgccatttatgcgagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaatt

SEQ ID NO: 2 - DNA, 367 bp, Homo sapiens
gcatagtgttaccatcaaccaccttaacttcatttttcttattcaatacctaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaacctaaaatgaaatgaaatcagacccttgtcttacaccataaacaaaaataaatttgaatgggttaaagaattaaactaagacctaaaaccataaaaatttttaaagaaatcaaaagaagaaaattctaatattcatgttgcagccg

SEQ ID NO: 3 - DNA, 188 bp, Homo sapiens
ttttttgaatttgatatgagaagcaaaggcaacaaaaggaaaaataaagaagtgaggctacatcaaactaaaaaatttccacacaaaaaagaaaacaatgaacaaatgaaaggtgaaccatgaaatggcatatttgcaaaccaaatatttcttaaatattttggttaatatccaaaatatataagaaa

SEQ ID NO: 4 - DNA, 295 bp, Homo sapiens
cacagatgattcaataacaaacaaaaaattaaaaataggaaaataaaaaaattaaaaagaagaaaatcctgccatttatgcgagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaatt

SEQ ID NO: 5 - DNA, 2286 bp, Mus sp.
gaattctgaatgagagactaaaagcttatgaactgtggctttcctttgtcagtgggactctaagaatgagttggggacaaaagagataggaatggctttaaaggtgactagttgaactgataaagtaaatgaactgaggaaaaaaaatatcactcaagatgacaggctaaaaccttgagaagtcctgctggaagaggaaactgggtgatcgggaggggtgctggattgatgaatctaaagagttgcgtggtcaaagggtcatgttaaaatttagatgctgccttcactgctgggttcttctctctattccaggagccagttgttcatatacactttggtctatgaattttatagacttgtccaagtgctgtgtatgagtttacatacacagtcatttatagccatatgacatttttattacagacctggcttcagattgtgcacattattcttcaaccagctcttacttatcctttatagctttactttaagcatagtacttgaagtgatgatgaggccatattaaagaaaggtccttttcactcccctcgtgccccacactctcttctccttcaacctgagctttggcctcctggtgaaagtatcaaagagaaaaagactgggagggtagaatggagattgggccctggcctgatggaaaccattaagtttagccagacaattcgagtaaggtgttgctatcaactgggctaataacgagccaggtgtggaagcactatctgaagggcactggttcagtacccactccgacataccaaaggggagcttaaaactttaagagaattatgcctttcaagcactatttcctgagccacttctgctttccacagagtcaacacatgaaactaaagagcccagtgagctctgtttttgagagggcatccatggggaaataattcctatctgtctgccatctatctgtttttcttgtattttgcctcatgatccagaaggctgggttgctagaatattccaggcctgtgtgtgtgtgtgtgtgtgtgtgtgtgtgtgtgtgtgtgtgtgtgtgtgtgtgttagagatggtctcatagaacactgggaacaggttatgttgtcagcgtgacttgacacaggatgttaggctgatcatttatttccttaaaggcggtgtttgccaagtttcctcactgtgcagttattactttccccttgcagagtctttctgggggagcaaaccgttaactacagctctctcaccaaagtgagacatgaaaggtactaagcttctggaaggggaacagtcctggttcctttcattagagaatggtattgcaaagatctgtttactggctctgcccctcgttagtgagggctcactgtttatacaactttgtagagtagaactaagaaacataaagtatgaagacatatgcacacacatcatcaccatcatatctctctctctctccctctccctctccctctccctccccccccctctctctctctctctctctctctcatctatctatgcacataagagtttcaactgataattctgactctctttcagagccacaaggctcagtccaactttcctcttgctgatctataagtgtccttttgattgtgaggacttgtcacccactatctgccctcagatatatatttatattatatatttgtttaagcccaggatatatgttaaggagcttcagaattattgacctcatacttccataagaaccatcaacaactaggatatagtacttacatggaatttcttttgtctttaaccttatagtttttaggcaaaatattgttttccaaagttacttaggtcaattctatttttttctttatattcttcagtgaggttatggtatagtttagtttgaatcacatttacataggatacaagacaaatttaggctgtcttttgaatgattctttggtttatactgcacagatcggtccattgtgtctttcctatatttcagtagagaagcctccgttgatagtataaaaaggtagcagagagggtagcaagcattcttctcccagtgcactctctgctaatgttatctagggcgttcattcccatctctccacaaactccatctcaattctctctcagtcaccaggagtggcatgcctacttcatcccaggtatttgaagactatttgtctgtcactctcttgggcctttttagtgcatcctatcttgctttcttcagtgtgttcacactgtgtttcctcaagttggtattgcctttccaggaatataaccacttcccttttctta

SEQ ID NO: 6 - DNA, 11 bp, Artificial
  MAZ sequence
gggggaggggg

SEQ ID NO: 7 - DNA, 11 bp, Artificial
  ZAM sequence
ccccctccccc

SEQ ID NO: 8 - DNA, 1185 bp, Homo sapiens
gattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccggtgggaagaaagacctctataggacaggacttctcagaaacagatgttttggaagagatgggaaaaggttcagtgaagacctgggggctggattgattgcagctgagtagcaaggatggttcttaaggaagggaaagtgttccaagctttaggaattcaaggtttagtcaggtgtagcaattctattttattaggaggaatactatttctaatggcacttagcttttcacagcccttgtggatgcctaagaaagtgaaattaatcccatgccctcaagtgtgcagattggtcacagcatttcaagggagagacctcattgtaagactctgggggaggtggggacttaggtgtaagaaatgaatcagcagaggctcacaagtcagcatgagcatgttatgtctgagaaacagaccagcactgtgagatcaaaatgtagtgggaagaatttgtacaacattaattggaaggcttacttaatggaatttttgtatagttggatgttagtgcatctctataagtaagagtttaatatgatggtgttacggacctaatgtttgtgtctcctcaaaattcacatgctgaatccccaactcccaactgaccttatctgtgggggaggcttttgaaaagtaattaggtttagatgagctcataagagcagatccccatcataaaattattttccttatcagaagcagagagacaagccatttctctttcctcccggtgaggacacagtgagaagtccgccatctgcaatccaggaagagaaccctgaccacgagtcagccttcagaaatgtgagaaaaaactctgttgttgaagccacccagtcttttgtattttgttatagcaccttgcactgagtaaggcagatgaagaaggagaaaaaaataagcttgggttttgagtggactacagaccatgtttatctcaggtttgcaaagctcccc

SEQ ID NO: 9 - DNA, 1185 bp, Homo sapiens
ttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaagggggctgaacccccttccctggattgcagcacagcagcgaggaaggggctcaacgaagaaaaagtgttccaagctttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacc

SEQ ID NO: 10 - DNA, 1178 bp, Homo sapiens
ctccatcttttactcttgtgttaaaaggaaaaagtgttcatgggctgagggatggagagaaacataggaagaaccaagagcttccttaagaaatgtatgggggcttgtaaaattaatgtggatgttatgggagaattccaggattccaaggaggatgatatgatggagaaaaatctttatcggggtgggaaaatggttaattaagtggacagagactcctaggcagtttttactgcaccggggaaagaaggagctgttagtggtacctgagaaagcagatttgtggtacatgtcacttttcattaaaaacaaaaacaaaacaaaacaaaacttcatagatatccaagatataggctagaattactattttaatttactcttatttacattttgaagtagctagcttgtcacatgttttatgaaattgatttggagataagatgagtgtgtatcaacaatagcctgctctttccatgaaggattccattatttcatgggttagctgaagctaagacacatgatatcattgtgcattatcttctgatagaatgtaacatgcactaaaataaagttagagttaggacctgagtgggaaagtttttggagagtgtgatgaagactttccgtgggagatagaatactaataaaggcttaaattctaaaaccagcaagctagggcttcgtgacttgcatgaaactggctctctggaagtagaagggagagtaagacatacgtagaggactaggaaagaccagatagtacagggcctggctacaaaaatacaagcttttactatgctattgcaatactaaacgataagcattaggatgttaagtgactcaggaaataagattttgggaaaaagtaatctgcttatgtgcacaaaatggattcaagtttgcagataaaataaaatatggatgatgattcaaggggacagatacaatggttcaaacccaagaggagcagtgagtctgtggaatttgaaggatggacaaaggtggggtgagaaagacatagtattcgactgactgtgggagatgagaaggaagaaggaggtgataaatgactgaaagctcccagactggtgaagataacaggaggaaaccatgcactgacctggtgactctcatgtgtgaagggtagagggatattaacagatttactttttaggaag

SEQ ID NO: 11 - DNA, 69 bp, Mus sp.
gaagtaaagagttagagtatggtgagaaattataaaccatcaaagaaaaaaatacaggacccataaagg

SEQ ID NO: 12 - DNA, 359 bp, Homo sapiens
ttacccagagtgcaggtgtgtggagatccctcctgccttgacattgagcagccttagagggtgggggaggctcaggggtcaggtctctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagtggggctgtctcagggtagctgggcacggtgttcccttgagtgggggtgtagtgggtgttcctagctgccacgcctttgccttcacctatggga

SEQ ID NO: 13 - DNA, 2866 bp, Homo sapiens
atgggggtgcacggtgagtactcgcgggctgggcgctcccgcccgcccgggtccctgtttgagcggggatttagcgccccggctattggccaggaggtggctgggttcaaggaccggcgacttgtcaaggaccccggaagggggaggggggtggggcagcctccacgtgccagcggggacttgggggagtccttggggatggcaaaaacctgacctgtgaaggggacacagtttgggggttgaggggaagaaggtttgggggttctgctgtgccagtggagaggaagctgataagctgataacctgggcgctggagccaccacttatctgccagaggggaagcctctgtcacaccaggattgaagtttggccggagaagtggatgctggtagctgggggtggggtgtgcacacggcagcaggattgaatgaaggccagggaggcagcacctgagtgcttgcatggttggggacaggaaggacgagctggggcagagacgtggggatgaaggaagctgtccttccacagccacccttctccctccccgcctgactctcagcctggctatctgttctagaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacggtgagaccccttccccagcacattccacagaactcacgctcagggcttcagggaactcctcccagatccaggaacctggcacttggtttggggtggagttgggaagctagacactgcccccctacataagaataagtctggtggccccaaaccatacctggaaactaggcaaggagcaaagccagcagatcctacggcctgtgggccagggccagagccttcagggacccttgactccccgggctgtgtgcatttcagacgggctgtgctgaacactgcagcttgaatgagaatatcactgtcccagacaccaaagttaatttctatgcctggaagaggatggaggtgagttcctttttttttttttttcctttcttttggagaatctcatttgcgagcctgattttggatgaaagggagaatgatcgagggaaaggtaaaatggagcagcagagatgaggctgcctgggcgcagaggctcacgtctataatcccaggctgagatggccgagatgggagaattgcttgagccctggagtttcagaccaacctaggcagcatagtgagatcccccatctctacaaacatttaaaaaaattagtcaggtgaggtggtgcatggtggtagtcccagatatttggaaggctgaggcgggaggatcgcttgagcccaggaatttgaggctgcagtgagctgtgatcacaccactgcactccagcctcagtgacagagtgaggccctgtctcaaaaaagaaaagaaaaaagaaaaataatgagggctgtatggaatacattcattattcattcactcactcactcactcactcattcattcattcattcattcaacaagtcttattgcataccttctgtttgctcagcttggtgcttggggctgctgaggggcaggagggagagggtgacatgggtcagctgactcccagagtccactccctgtaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggtgagtaggagcggacacttctgcttgccctttctgtaagaaggggagaagggtcttgctaaggagtacaggaactgtccgtattccttccctttctgtggcactgcagcgacctcctgttttctccttggcagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccagcaatgacatctcaggggccagaggaactgtccagagagcaactctgagatctaaggatgtcacagggccaacttgagggcccagagcaggaagcattcagagagcagctttaaactcagggacagagccatgctgggaagacgcctgagctcactcggcaccctgcaaaatttgatgccaggacacgctttggaggcgatttacctgttttcgcacctaccatcagggacaggatgacctggataacttaggtggcaagctgtgacttctccaggtctcacgggcatgggcactcccttggtggcaagagcccccttgacaccggggtggtgggaaccatgaagacaggatgggggctggcctctggctctcatggggtccaagttttgtgtattcttcaacctcattgacaagaactgaaaccaccaatatgactcttggcttttctgttttctgggaacctccaaatcccctggctctgtcccactcctggcagcagtgcagcaggtccaggtccgggaaacgaggggtggagggggctgggccctacgtgctgtctcacacagcctgtctga

SEQ ID NO: 14 - DNA, 23 bp, Artificial
  oligonucleotide primer designated e2f
tggcctggctcacctggacaacc

SEQ ID NO: 15 - DNA, 19 bp, Artificial
  oligonucleotide primer designated e3r
atccagatgctcaaggccc

SEQ ID NO: 16 - DNA, 24 bp, Artificial
  oligonucleotide primer designated RTf/VF
caggaaactattactcaaagggta

SEQ ID NO: 17 - DNA, 23 bp, Artificial
  oligonucleotide primer designated VR/pAR
cttgaatccttttctgagggatg

SEQ ID NO: 18 - DNA, 24 bp, Artificial
  oligonucleotide primer designated RTr
agaaaataccgcatcaggcgccat

SEQ ID NO: 19 - DNA, 23 bp, Artificial
  oligonucleotide primer designated TAR3′
gagctttattgaggcttaagcag

SEQ ID NO: 20 - DNA, 21 bp, Artificial
  oligonucleotide primer designated TERM5′
gcatagtgttaccatcaacca

SEQ ID NO: 21 - DNA, 21 bp, Artificial
  oligonucleotide primer designated TERM3′
tttcctgattctcccaccccc

SEQ ID NO: 22 - DNA, 21 bp, Artificial
  oligonucleotide primer designated PMF
gcttcaggtacaactggccac

SEQ ID NO: 23 - DNA, 21 bp, Artificial
  oligonucleotide primer designated PMR
ggagcacactcacctgcccac

SEQ ID NO: 24 - DNA, 21 bp, Artificial
  oligonucleotide primer designated EP5′/EPf′
aagctgtgacttctccaggtc

SEQ ID NO: 25 - DNA, 21 bp, Artificial
  oligonucleotide primer designated EP3′/EPr′
tggtttcagttcttgtcaatg

SEQ ID NO: 26 - DNA, 21 bp, Artificial
  oligonucleotide primer designated E5′
atgggggtgcacggtgagtac

SEQ ID NO: 27 - DNA, 22 bp, Artificial
  oligonucleotide primer designated E3′
tcagacaggctgtgtgagacag

SEQ ID NO: 28 - DNA, 23 bp, Artificial
  oligonucleotide primer designated APR
aaaggcagggattcctctgagcc

SEQ ID NO: 29 - DNA, 24 bp, Artificial
  oligonucleotide primer designated APF
ccctaaggaacacaaatttcttta

SEQ ID NO: 30 - DNA, 22 bp, Artificial
  oligonucleotide primer designated pAF
ccacaagtatcactaagctcgc

SEQ ID NO: 31 - DNA, 23 bp, Artificial
  oligonucleotide primer designated R
aactagctcttcatttctttatg

SEQ ID NO: 32 - DNA, 22 bp, Artificial
  oligonucleotide primer designated F
ccttgggaaaatacactatatc

SEQ ID NO: 33 - DNA, 24 bp, Artificial
  oligonucleotide primer designated 4.5
ttgtgggccagggcattagccaca

SEQ ID NO: 34 - DNA, 22 bp, Artificial
  oligonucleotide primer designated SPAf
ccttgggaaaatacactatatc

SEQ ID NO: 35 - DNA, 23 bp, Artificial
  oligonucleotide primer designated I2r
ctatgacatgaacttaaccatag

SEQ ID NO: 36 - DNA, 21 bp, Artificial
  oligonucleotide primer designated e1f
actcctgaggagaagtctgcc

SEQ ID NO: 37 - DNA, 22 bp, Artificial
  oligonucleotide primer designated e2r
tttcttgccatgacccttcacc

SEQ ID NO: 38 - DNA, 20 bp, Artificial
  oligonucleotide primer designated I1r
tcagtgcctatcagaaaccc

SEQ ID NO: 39 - DNA, 22 bp, Artificial
  oligonucleotide primer designated e3f
ccacaagtatcactaagctcgc

SEQ ID NO: 40 - DNA, 24 bp, Artificial
  oligonucleotide primer designated U35′
ttacggttcctggccttttgctgg

SEQ ID NO: 41 - DNA, 48 bp, Artificial
  oligonucleotide primer designated U3T7
taatacgactcactatagggaggtttcctgtgtgaaattgttatccgc

SEQ ID NO: 42 - DNA, 6 bp, Homo sapiens
aataaa

SEQ ID NO: 43 - DNA, 6 bp, Homo sapiens
attaaa

SEQ ID NO: 44 - RNA, 6 nt, Homo sapiens
aaaaaa

SEQ ID NO: 45 - DNA, 349 bp, Artificial
  RZMAZ4 terminator sequence
cctgtcaccggatgtgttttccggtctgatgagtccgtgaggacgaaacaggccttgggaaaatacactatatcttaaactccatgaaagaaggtgaggctgcaaacagctaatgcacattggcaacagccctgatgcctatgccttattcatccctcagaaaaggattcaagggccgcgccgtcgacctggccttgggggagggggaggccagaatgagagctcctggccttgggggagggggaggccagaatgactcgacctggccttgggggagggggaggccagaatgagagctcctggccttgggggagggggaggccagaatgactcgaggaattcccatgca

SEQ ID NO: 46 - DNA, 176 bp, Artificial
  MAZ4
ggccgcgccgtcgacctggccttgggggagggggaggccagaatgagagctcctggccttgggggagggggaggccagaatgactcgacctggccttgggggagggggaggccagaatgagagctcctggccttgggggagggggaggccagaatgactcgaggaattcccatgca

SEQ ID NO: 47 - DNA, 52 bp, Artificial
  Hammerhead ribozyme
cctgtcaccggatgtgttttccggtctgatgagtccgtgaggacgaaacagg

SEQ ID NO: 48 - DNA, 21 bp, Artificial
  oligonucleotide primer designated COTC3′
cggctgcaacatgaatattag

SEQ ID NO: 49 - DNA, 23 bp, Artificial
  oligonucleotide primer designated YFPf
accatgggatccagtgagcaagg

SEQ ID NO: 50 - DNA, 22 bp, Artificial
  oligonucleotide primer designated RABr
tcaagacgatgagcaacaaggc

(19) United States
(12) Patent Application Publication
(10) Pub. No.: US 2011/0195451 A1
(43) Pub. Date: Aug. 11, 2011

(54) Protein Expression

(75) Inventors: Nicholas Jarvis Proudfoot, Oxford (GB); Michael John Dye, Oxford (GB); Steven James West, Barnton (GB)

(73) Assignee: ISIS INNOVATION LIMITED, Summertown, Oxford (GB)

(21) Appl. No.: 12/996,900

(22) PCT Filed: Jun. 9, 2009
(86) PCT No.: PCT/GB09/50645

§ 371 Date: Feb. 14, 2011

(30) Foreign Application Priority Data
Jun. 10, 2008 (GB) 0810567.8
Jun. 30, 2008 (GB) 0811893.7
Feb. 11, 2009 (GB) 0902222.9

(51) Int. Cl. C12P 21/00; C07H 21/04; C07K 2/00; C12N 5/071; C12N 5/07; C12N 5/04; C12N 1/20

(52) U.S. Cl. 435/69.1; 536/23.5; 530/300; 435/325; 435/348; 435/410; 435/252.1; 435/255.1

(57) ABSTRACT

An isolated DNA molecule having a sequence which comprises in a 5′ to 3′ direction (i) one or more promoter elements, (ii) the geneof interest, and (iii) a poly-adenylation 5 signal, and (iv) a terminator element, and expressing the geneof interest incorporated into the DNA molecule in an expression system, and use of said molecule to enhance expression of a gene of interest.

### FIELD OF THE INVENTION

The present invention relates to a method of enhancing expression from genes contained in a DNA construct or integrated into chromosomal locations in cells.

### BACKGROUND OF THE INVENTION

The transcription cycle consists of three phases: transcriptional initiation, which involves the association of RNA polymerase with the DNA template; transcriptional elongation, in which the tight association of polymerase with the DNA template is maintained as the polymerase progresses through the body of the gene; and finally transcription termination (hereafter: termination), when dissociation of the polymerase and DNA template takes place.

Transcription by RNA polymerase II (Pol II) is the first step in the expression of protein-coding genes and can be controlled by a wide range of cues that often regulate Pol II initiation. In addition, both the elongation rate of Pol II and the efficiency and accuracy of pre-mRNA processing can determine gene expression levels.

Transcriptional termination of mammalian RNA polymerase II (Pol II) is an essential but little-understood step in protein-coding gene expression. Mechanistically, termination by all DNA-dependent RNA polymerases can be reduced to two steps, namely release of the RNA transcript and release of the DNA template. However, to-date, transcriptional termination has often been considered a largely irrelevant process, only serving as a means to recycle polymerases or to prevent interference of downstream promoters. In particular, it is not considered when designing systems for in vivo expression of protein in cell lines or tissues.

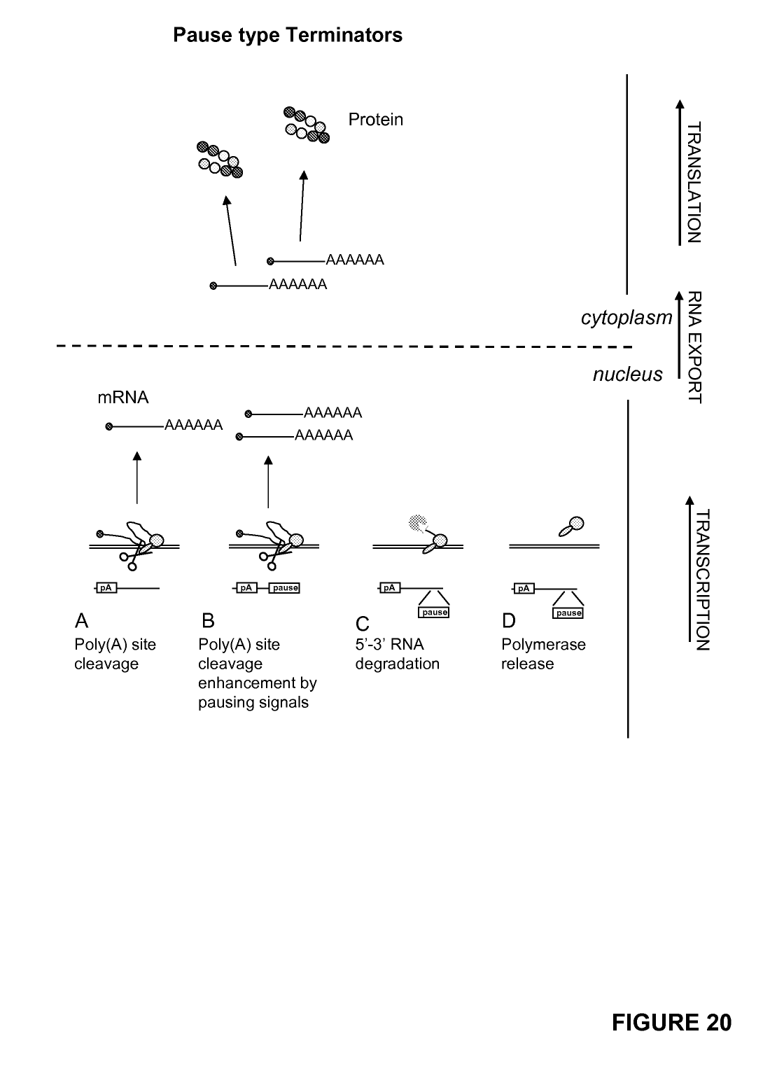

One mechanism of transcriptional termination proceeds via cessation of RNA synthesis followed by Pol II-DNA dissociation. The 3′ end of protein encoding genes, with the exception of replication-dependent histone genes, is defined by a poly(A) signal, which is required for efficient 3′ end formation and rendering Pol II termination competent. It consists of an upstream, largely invariant, hexanucleotide sequence (AATAAA) followed by a more variable GU-rich tract. The poly(A) signal provides a binding platform for various trans-acting proteins, which participate in cleavage of the primary transcript. The actual site of transcript cleavage lies between the AAUAAA and GU-rich elements, commonly after a CA di-nucleotide. The upstream product of cleavage is subject to a polyadenylation reaction, which acts to protect the transcript from exonucleases, promote its export to the cytoplasm and enhance its translation. This is shown in FIG. 20 and described further below.

A functional poly(A) signal is required for Pol II termination and dedicated termination signal sequences located downstream of the poly(A) signal, in mammalian genes, are required for efficient termination. Examples of dedicated termination signals include cotranscriptional cleavage (CoTC) and pause site termination signals as well as alternative exonuclease entry points^{1, 5}. These termination signals are required for release by Pol II of the DNA template.

The mechanism of poly(A) signals and pause type terminators are shown in FIG. 20

Poly(A) Signals

FIG. 20A. Transcription and pre-mRNA processing. RNA polymerase (complete circle) produces an RNA transcript (the pre-messenger RNA (pre-mRNA) indicated by a single line) as it processes along the DNA template (parallel lines). Upon transcribing the poly(A) signal (pA in the lower line diagram) the RNA is cleaved at the corresponding poly(A) cleavage site in the RNA (scissors denote pre-mRNA cleavage at the poly(A) cleavage site). The cleaved pre-mRNA is further processed by the addition of a polyadenylate ‘tail’ (AAAAAA in the figure) to become mature messenger RNA (mRNA). This mRNA is subsequently exported to the cytoplasmic compartment of the cell where it is translated within ribosome complexes into proteins which are shown here as joined circles.

NB. The poly(A) signal which is a pre-mRNA processing signal is sometimes referred to as a chain terminator or terminator in the literature.

FIG. 20C. Degradation of the downstream product of poly(A) site cleavage. Following cleavage at the poly(A) site the polymerase continues transcribing and producing an RNA transcript. This transcript is degraded by 5′-3′ RNA exonucleases (circle with segment removed).

FIG. 20D. Eventually when all of the downstream product of poly(A) site cleavage is degraded polymerase releases from the DNA template.

Pause Type Terminators

FIG. 20B. Pause terminators (or pause elements) can enhance the efficiency of pre-mRNA processing at the poly(A) site. The positioning of pause elements (pause in the lower line diagram) past the poly(A) site can enhance processing of the pre-mRNA at the poly(A) site and thus lead to an increase in the abundance of mature mRNA, as indicated by the 2 mRNAs above the diagram. This increase in the level of mRNA is reflected in the cytoplasm so there is an increase in protein level.

Several pause elements were described in the literature from 1985 to 2000, for example the MAZ terminator sequence and the human β-actin terminator sequence^{6, 16}. The maximum increase in protein level due to the inclusion of transcription pause elements is 2-3 fold and is highly dependent on the poly(A) site used.

CoTranscriptional Cleavage (CoTC) Type Terminators

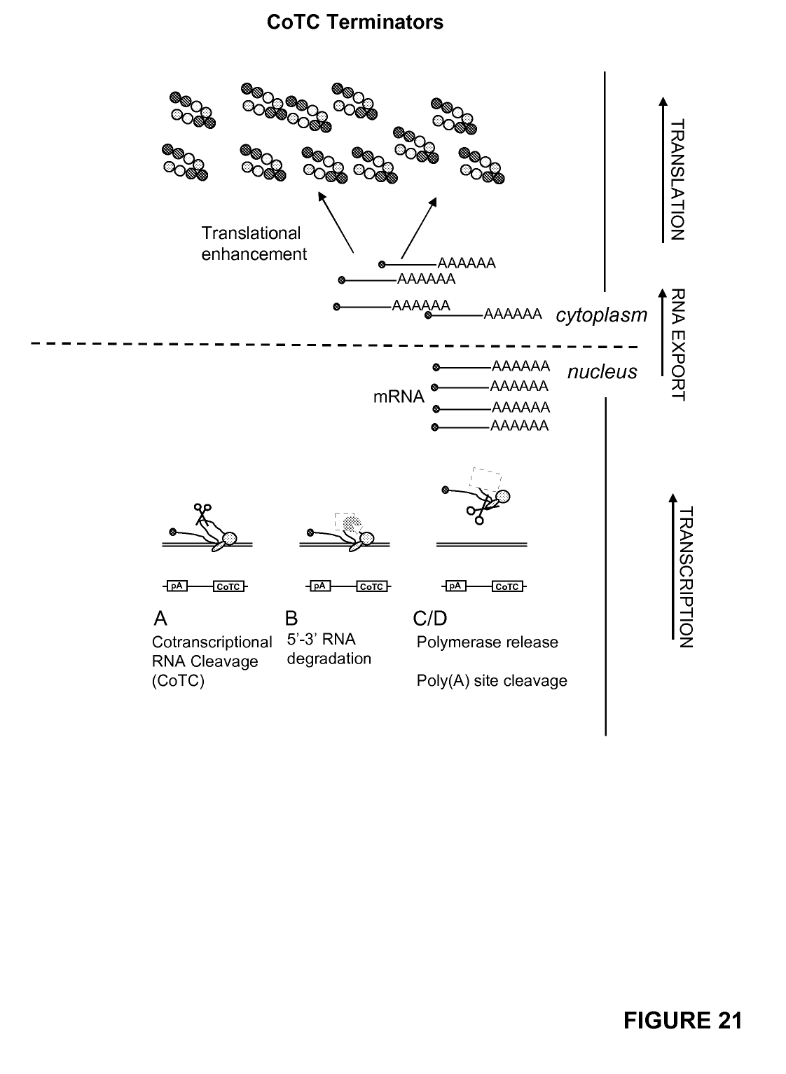

The mechanism of CoTC terminators is shown in FIG. 21, which is discussed below. It is currently known that the initial cleavage of the pre-mRNA is made whilst the polymerase continues transcribing and producing an RNA transcript, at positions downstream of the poly(A) site within the RNA transcript encoded by the DNA CoTC element. The transcript is then degraded by 5′-3′ RNA exonucleases, whilst the pre-mRNA, not yet cleaved at the poly(A) site, remains attached to the transcribing polymerase.

Possibly the most fully characterized Pol II CoTC type terminator sequence is that located in the 3′ flanking region of the human β-globin gene and transcripts of the β-globin terminator element are co-transcriptionally cleaved by an as yet uncharacterized activity termed cotranscriptional cleavage (CoTC)^{1, 5, 6}.

Another terminator sequence that mimics a CoTC terminator sequence comprises a highly efficient self-cleaving ribozyme RNA molecule with MAZ pause sequences downstream thereof².

Known DNA constructs comprising terminator sequences include:
human β-globin gene—β-globin terminator sequence and elements thereof^{1, 2} (CoTC type terminator);
human ε-globin gene—ε-globin terminator sequence ¹(CoTC type terminator);
human β-globin gene—MAZ4 terminator sequence ²(pause type terminator);
human β-globin gene—RZMAZ4 terminator sequence ^{2, 23}(CoTC type terminator);
human β-globin gene—5′RZ3′RZMAZ4 terminator sequence ²;
human β-globin gene—5′RZMAZ4 terminator sequence ²;
human β-globin gene—mouse serum albumin terminator sequence ⁵(CoTC type terminator);
human beta-actin gene—human beta-actin terminator sequence and human beta-globin—human beta-actin terminator sequence ⁶(pause type terminator);
mouse beta-major globin gene—mouse beta-major globin terminator sequence and elements thereof ¹⁴;
human gamma A globin gene—human gamma A globin terminator sequence and human gamma G globin gene—human gamma A globin terminator sequence ¹⁵(CoTC type terminator);
human gamma A globin gene—human gamma G globin terminator sequence and human gamma G globin gene—human gamma G globin terminator sequence ¹⁵(CoTC type terminator).

These constructs have been used solely as a tool to investigate mechanisms of transcription termination.

There are several known ways of enhancing the expression of genes. An established method is by using strong promoters, which result in more efficient initiation of the transcription reaction and, as such, a greater number of mRNAs. The strength of pre-mRNA processing signals also influences gene expression levels. In the nucleus, there is a constant competition between mRNA synthesis and degradation. Strong pre-mRNA processing signals result in more rapid splicing and cleavage and polyadenylation, both of which stabilise the resultant mRNA and enhance the possibility of it being exported to the cytoplasm where it can be translated into protein.

It is an object of the present invention to provide a further method of enhancing expression from a gene of interest.

### SUMMARY OF THE INVENTION

It has now been found that terminator sequences, particularly Pol II terminator sequences that encode a section of RNA that is cut co-transcriptionally, act to enhance the expression of genes. Surprisingly, not only is transcription of a gene of interest enhanced, but also translation of the resulting mRNA is enhanced.

Accordingly the present invention provides a method of enhancing expression of a gene of interest comprising providing an isolated DNA molecule having a sequence which comprises in a 5′ to 3′ direction (i) one or more promoter elements, (ii) the gene of interest, and (iii) a poly-adenylation signal, and (iv) a terminator element, and expressing the gene of interest incorporated into the DNA molecule in an expression system. Preferably the terminator sequence encodes a section of RNA that is cut co-transcriptionally. Because increased protein production is achieved by simply inserting a terminator sequence beyond the gene of interest, this invention is incredibly cheap and easy to implement. Further, the enhancing terminator sequence is positioned downstream of the gene of interest and so no alterations in the coding portion of the gene are required.

Advantageously, the amount of nuclear mRNA produced by this method is at least 2-fold greater than the amount produced by a method which is identical except that the DNA molecule does not contain a terminator element. Advantageously, the amount of cytoplasmic mRNA produced by this method is at least 3-fold greater than the amount produced by a method which is identical except that the DNA molecule does not contain a terminator element. Advantageously, the amount of protein produced by this method is at least 3-fold greater, and preferably 10-fold greater, than the amount produced by a method which is identical except that the DNA molecule does not contain a terminator element.

The present invention also provides an isolated DNA molecule having a sequence which comprises in a 5′ to 3′ direction (i) one or more promoter elements, (ii) a gene of interest, (iii) a poly-adenylation signal, and (iv) a terminator element, provided that the gene of interest is not the human β-globin gene, the human ε-globin gene, the human β-actin gene, the human gamma A globin gene, the human gamma G globin gene or the mouse β-major globin gene. Preferably the terminator sequence encodes a section of RNA that is cut co-transcriptionally.

In addition the present invention provides the use of one or more terminator elements in an isolated DNA molecule to enhance expression of a gene of interest, wherein the DNA molecule has a sequence which comprises in a 5′ to 3′ direction (i) one or more promoter elements, (ii) the gene of interest, (iii) a poly-adenylation signal, and (iv) one or more terminator elements. Preferably the terminator sequence encodes a section of RNA that is cut co-transcriptionally.

### BRIEF DESCRIPTION OF THE DRAWINGS

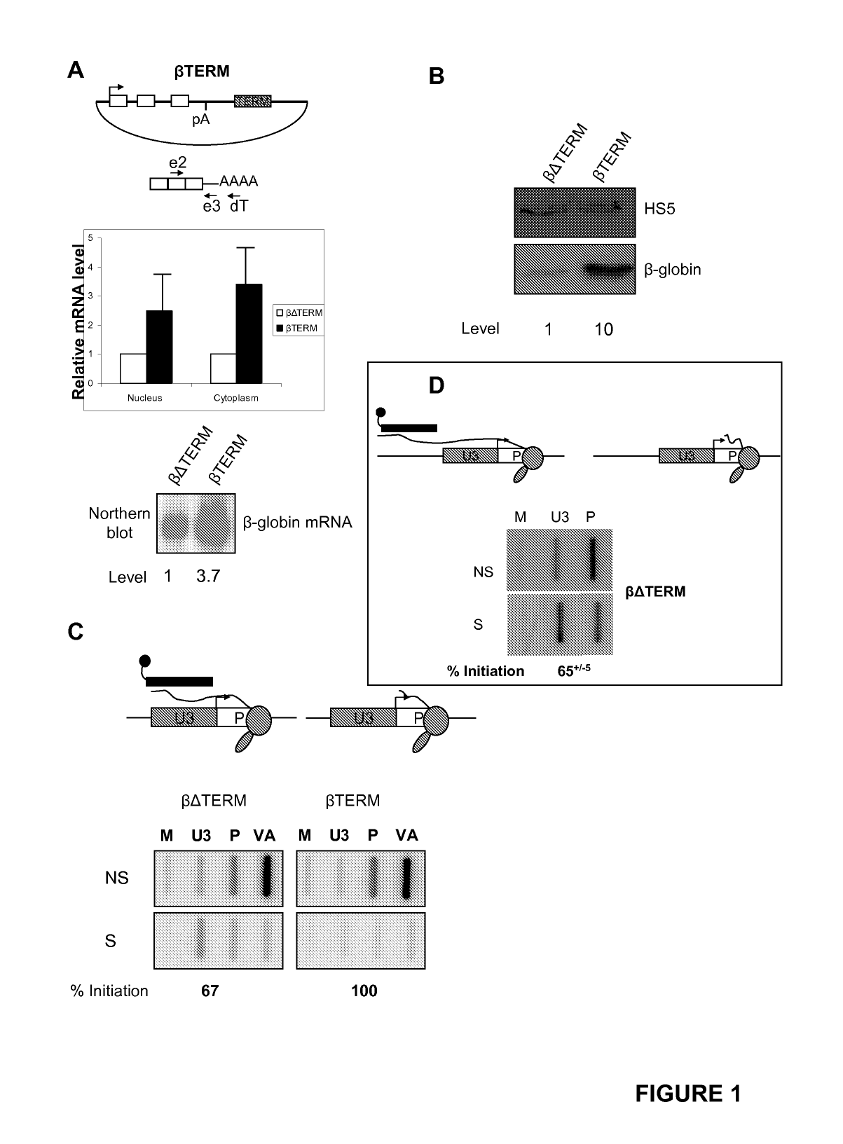

FIG. 1 shows:
A. Upper diagram shows βTERM. The HIV promoter (arrow), exons (white box), pA signal (pA) and terminator (TERM) are shown. The lower diagram shows spliced β-globin mRNA and the positions of the RT primer (dT) and subsequent primers (e2 and e3) that were used to detect it by real-time PCR. The graph shows the β-globin mRNA levels in the nuclei and cytoplasm of cells transfected with βΔTERM or βTERM after equalising to VA levels. β-globin mRNA levels were set at 1 for βΔTERM. The lower data panel displays northern blot analysis of cytoplasmic RNA from the same experiment.
B. Western blot analysis of HeLa cells transfected with βΔTERM or βTERM as well as the HS5 expression construct. β-globin and HS5 proteins were detected and are indicated.
C. Hybrid selection NRO analysis of HeLa cells transfected with βΔTERM or βTERM as well as VA. Diagrams show the HIV promoter U3 and P regions and the selection probe (black). This probe selects promoter (P) transcripts that result from read-through transcription (left diagram). Those that result from newly initiated Pol II are not selected (right diagram). Results for the experiment are shown on the left (βΔTERM) and right (βTERM). The top panels show transcripts not selected by the probe (NS) and the lower panels show selected (S) transcripts. NRO probes are shown above the relevant slot. % initiation is shown and was given a value of 100% for βTERM. M is an empty M13 vector that shows background signal. P signals were equalised to VA and βTERM was given a value of 100%.
D. RNA degradation does not influence interpretation of the hybrid selection NROs.

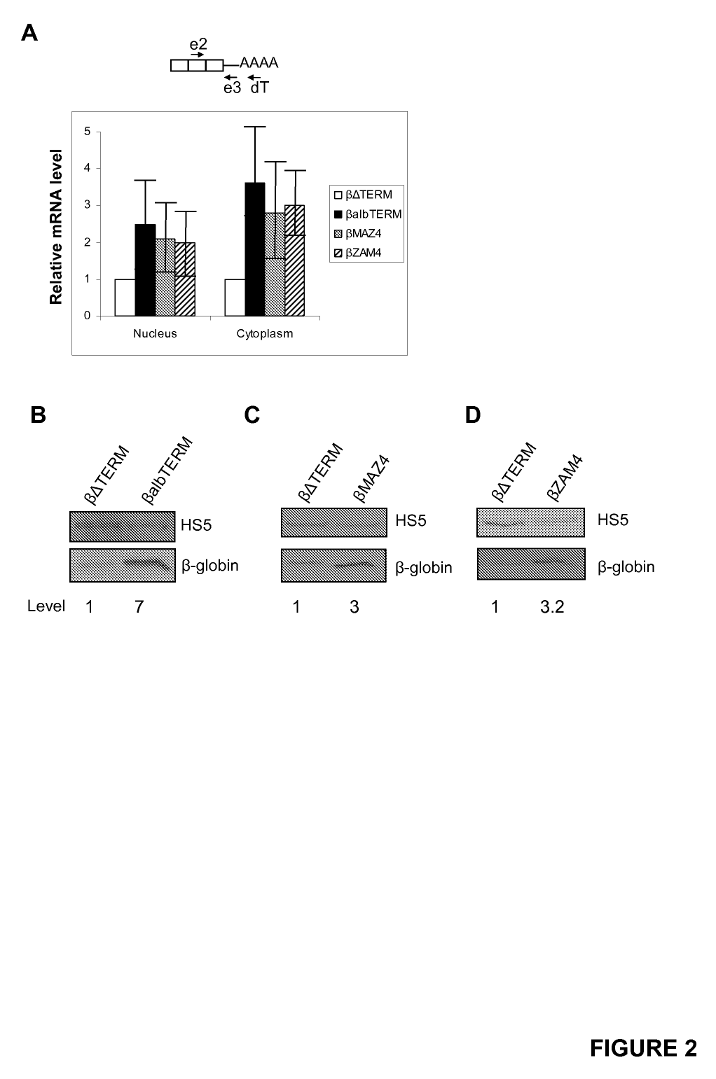

FIG. 2 shows:
A. Real time RT-PCR analysis of β-globin mRNA in the nucleus and cytoplasm of HeLa cells transfected with βΔTERM, βalbTERM, βMAZ4 or βZAM4. Primers are as in FIG. 1A. The graph shows β-globin mRNA values where those for βΔTERM are set to 1.
B. Western blotting analysis of HeLa cells transfected with βΔTERM or βalbTERM, together with HS5. β-globin and HS5 proteins are indicated.
C. Western blotting analysis of HeLa cells transfected with βΔTERM or βMAZ4, together with HS5. β-globin and HS5 proteins are indicated.
D. Western blotting analysis of HeLa cells transfected with βΔTERM or βZAM4, together with HS5. β-globin and HS5 proteins are indicated.

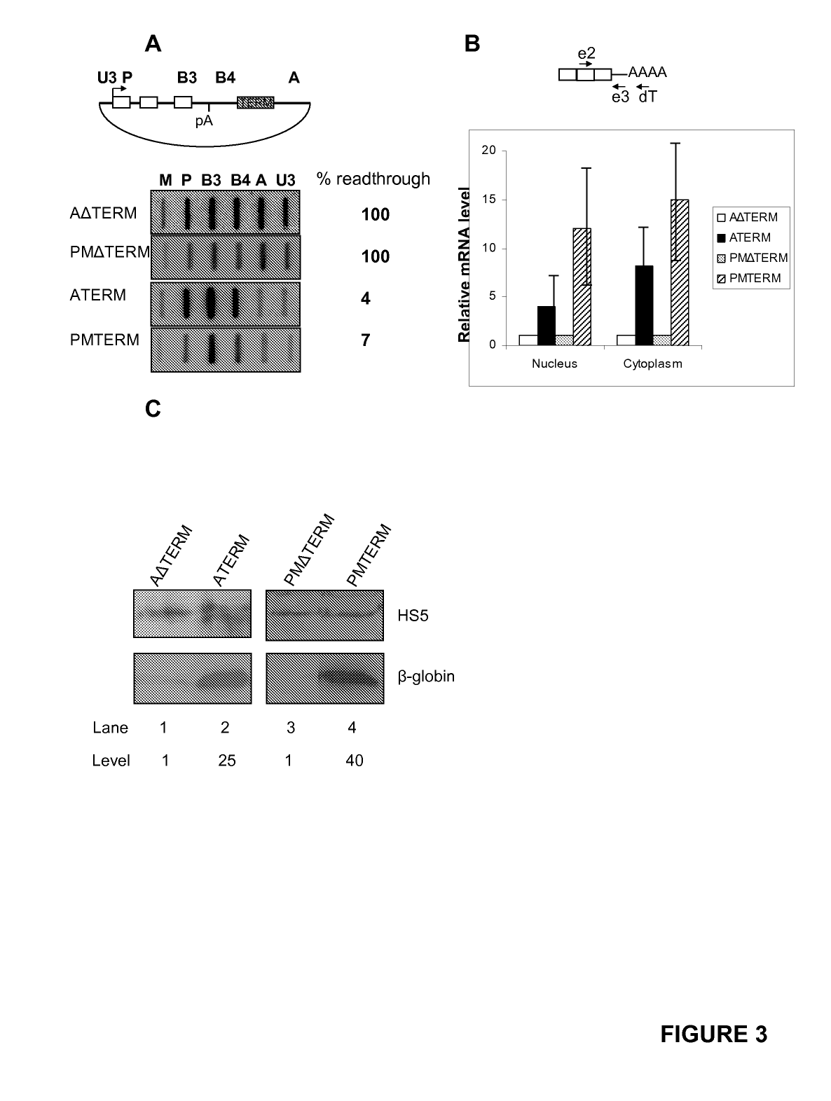

FIG. 3 shows:
A. NRO analysis of HeLa cells transfected with AΔTERM, ATERM, PMΔTERM or PMTERM. Probes are shown above the relevant slot and their position on the plasmid is shown in the diagram. The graph shows relative signals that were normalised to the B3 signal, which was set to 1. For comparison, values for βΔTERM and βTERM NRO's are shown.
B. Real-time RT-PCR analysis of nuclear and cytoplasmic β-globin mRNA from HeLa cells transfected with AΔTERM, ATERM, PMΔTERM or PMTERM. Primers were as in FIG. 1A. β-globin mRNA levels are shown on the graph, where the values obtained for the ΔTERM constructs are set at 1.
C. Western blotting analysis of β-globin protein in HeLa cells transfected with AΔTERM or ATERM (left blot) and PMΔTERM or PMTERM (right blot). β-globin and HS5 control proteins are indicated.

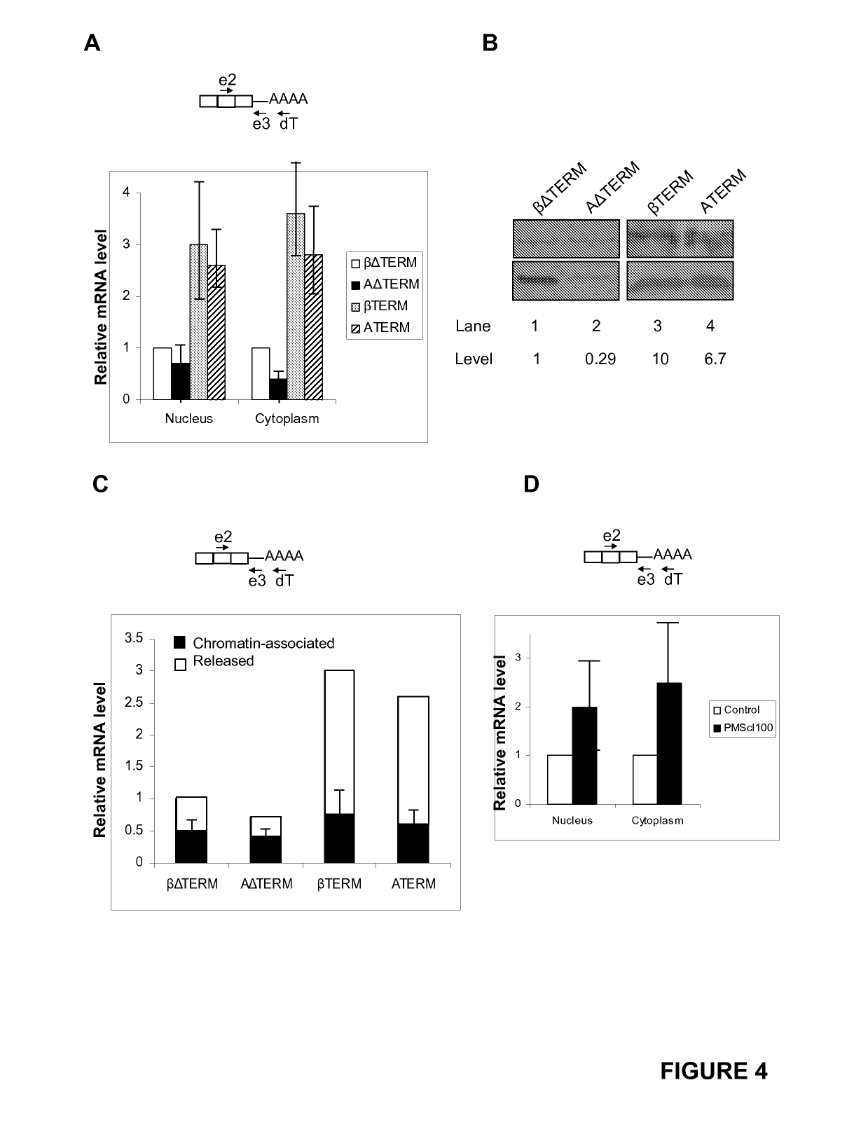

FIG. 4 shows:
A. Real time RT-PCR analysis of nuclear and cytoplasmic β-globin mRNA from HeLa cells transfected with βΔTERM, AΔTERM, βTERM or ATERM. Primers are as in FIG. 1A. Graph shows β-globin mRNA levels where βΔTERM samples are given a value of 1.
B. Western blot analysis of β-globin and HS5 proteins in HeLa cells transfected with βΔTERM, AΔTERM, βTERM or ATERM. The proteins are indicated. Lanes 1 and 2 were exposed for longer than lanes 3 and 4 due to the low level of β-globin protein when termination is inefficient.
C. Real-time RT-PCR analysis of chromatin-associated (black) and released (white) nuclear RNA isolated from HeLa cells transfected with βΔTERM, AΔTERM, βTERM or ATERM. Primers are as in FIG. 1A. The proportion of mRNA in each fraction is superimposed onto the levels of nuclear β-globin mRNA obtained from the experiment in A.
D. Real-time RT-PCR analysis of β-globin mRNA levels in the nucleus and cytoplasm of HeLa cells transfected with AΔTERM. Depletion of the PMScl100 subunit of the nuclear exosome enhances nuclear and cytoplasmic levels of β-globin mRNA.

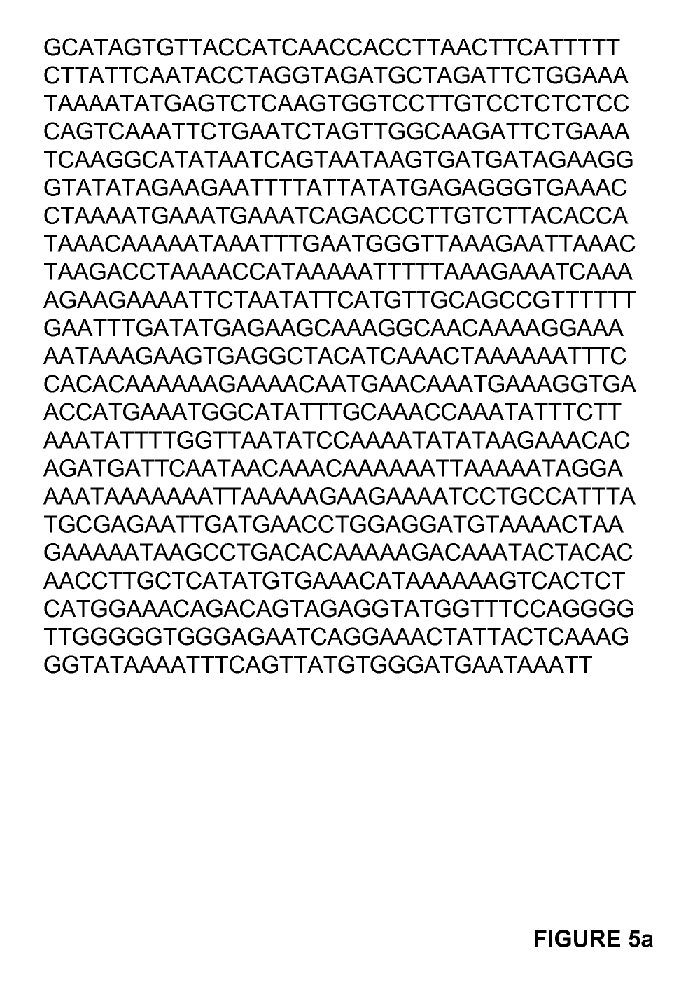

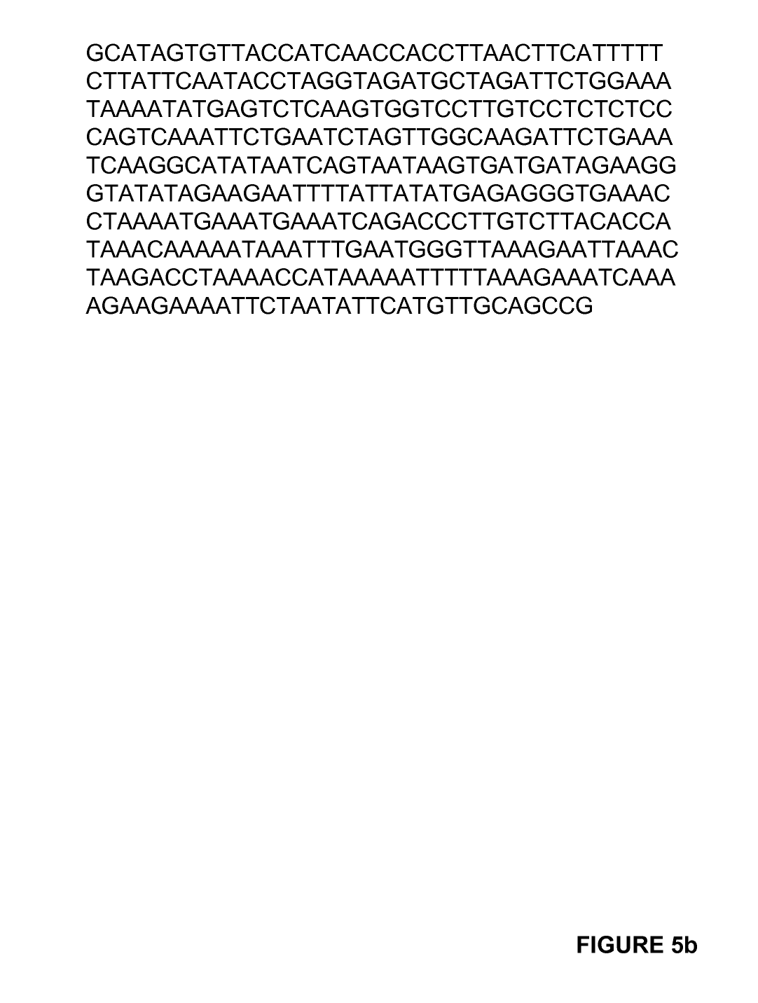

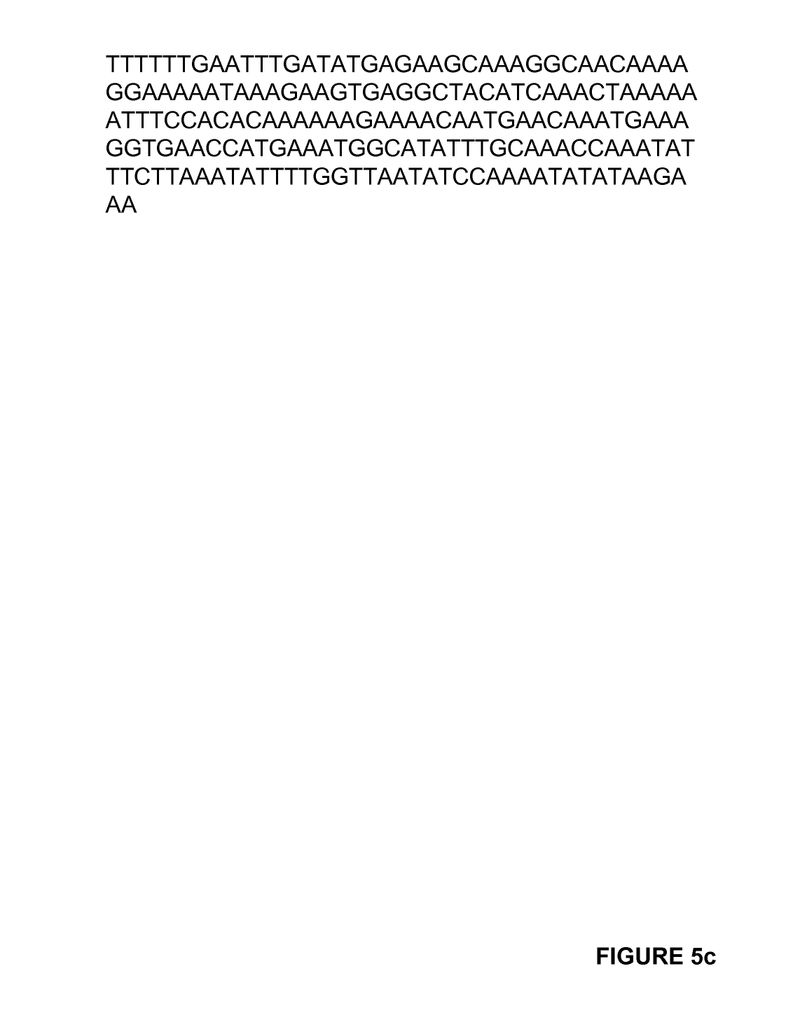

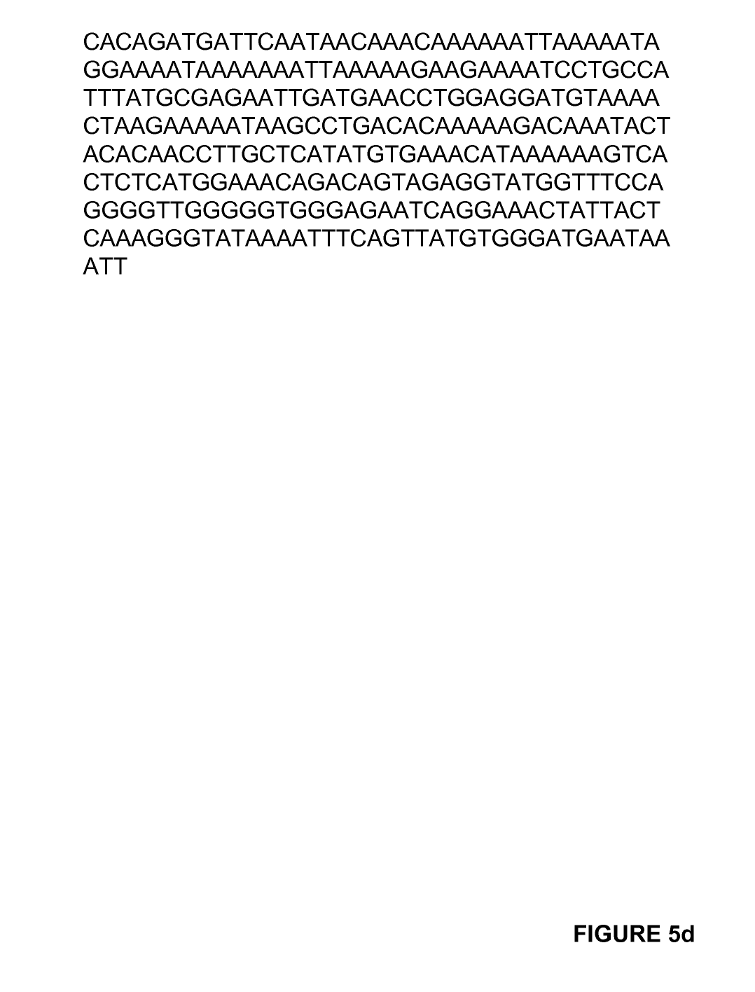

FIG. 5 shows:
A. The sequence of the human β-globin terminator region (GenBank sequence accession number U01317, nucleotides 64568-65421) (SEQ ID NO:1).
B. The sequence of element 8 of the human β-globin terminator sequence (GenBank sequence accession number U01317, nucleotides 64568-64938) (SEQ ID NO:2).
C. The sequence of element 9 of the human β-globin terminator sequence (GenBank sequence accession number U01317, nucleotides 64939-65126) (SEQ ID NO:3).
D. The sequence of element 10 of the human β-globin terminator sequence (GenBank sequence accession number U01317, nucleotides 65127-65421) (SEQ ID NO:4).

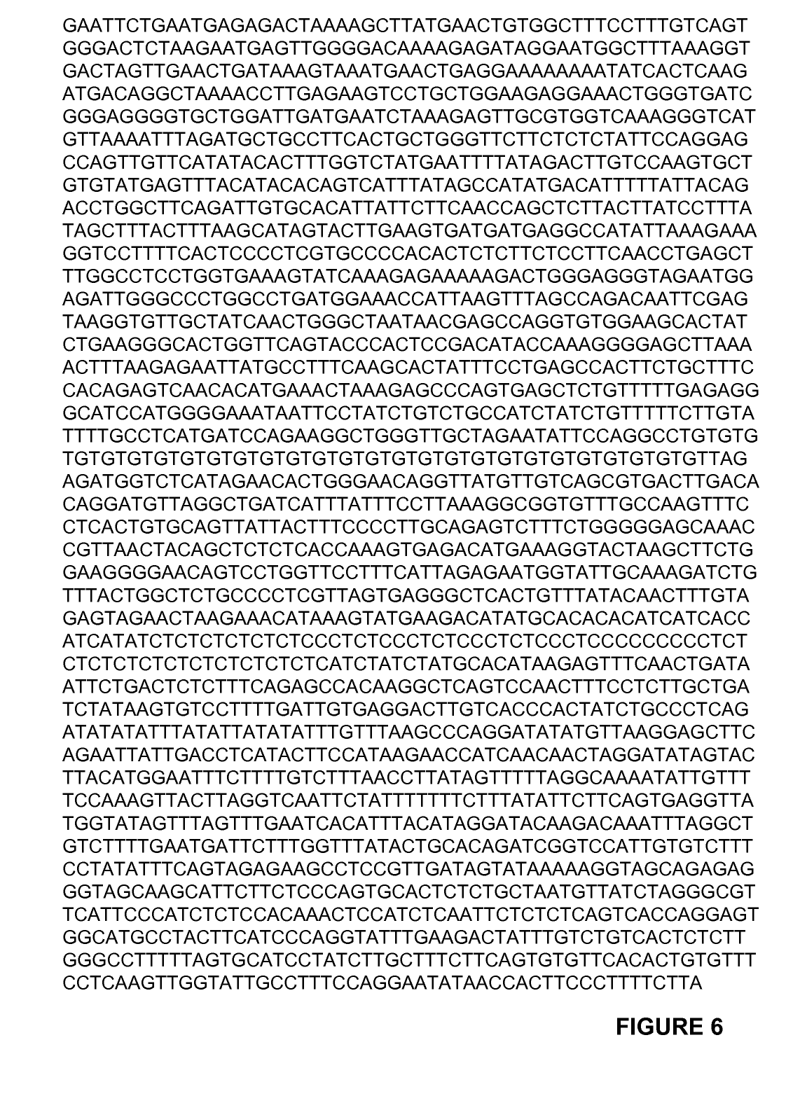

FIG. 6 shows:
The sequence of the mouse albumin terminator sequence (SEQ ID NO:5).

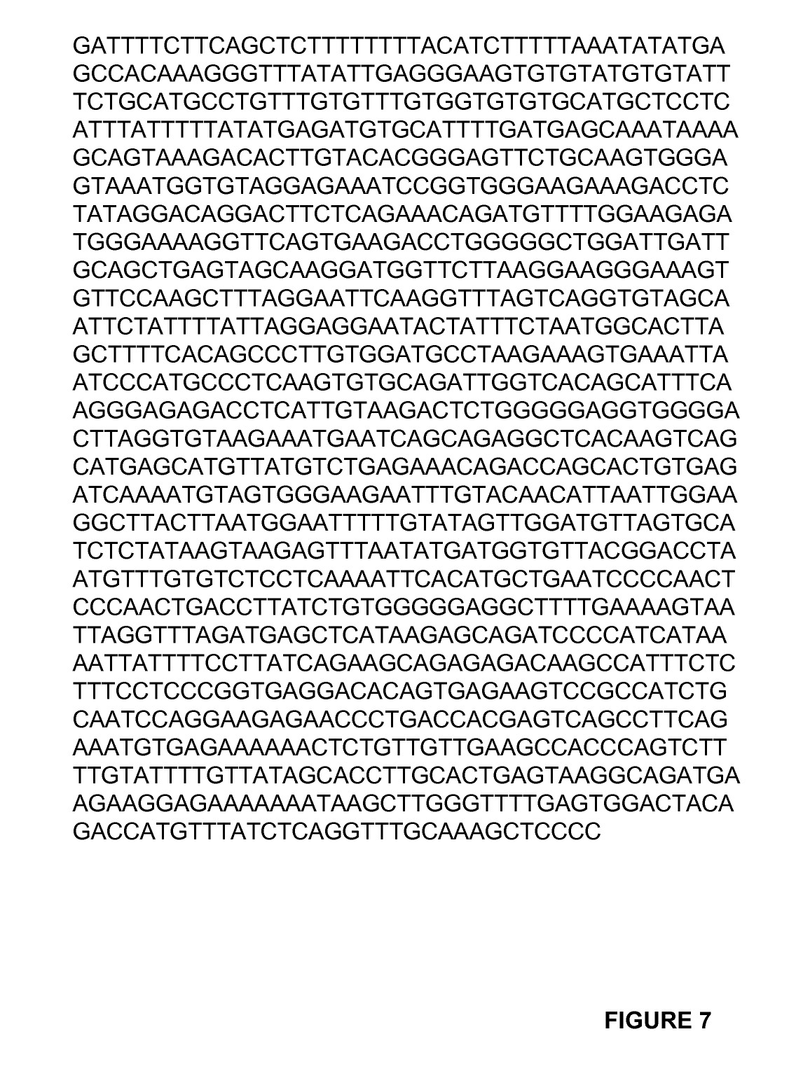

FIG. 7 shows:
The sequence of the human gamma A globin terminator sequence (SEQ ID NO:8).

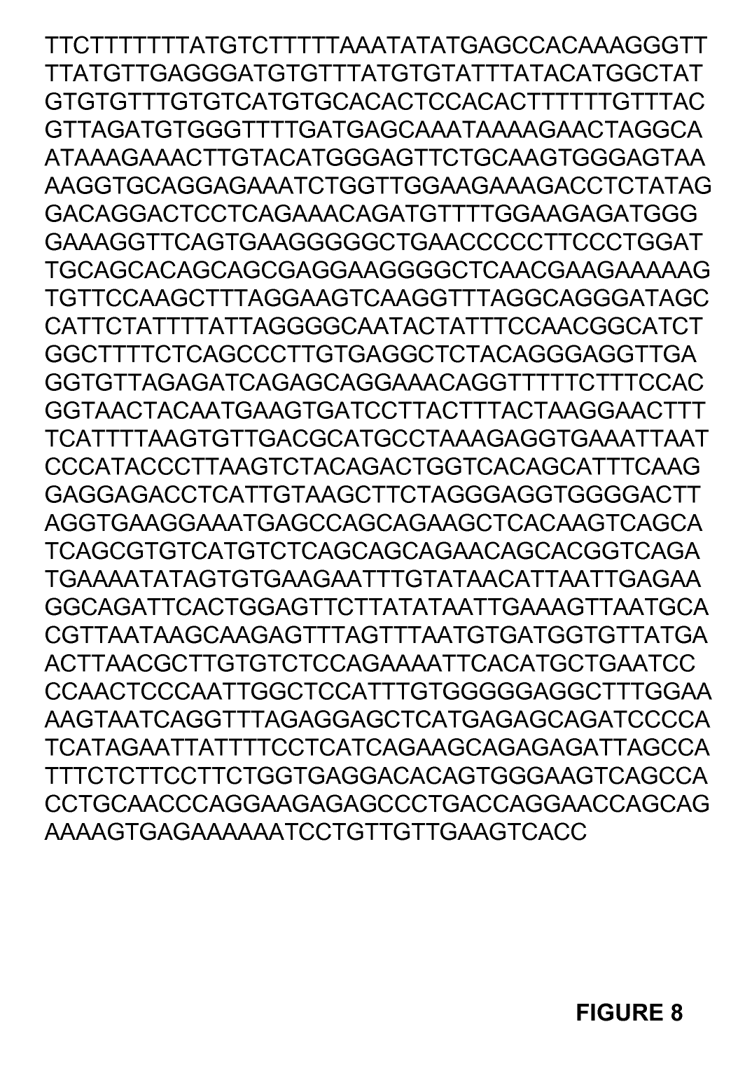

FIG. 8 shows:
The sequence of the human gamma G globin terminator sequence (SEQ ID NO:9).

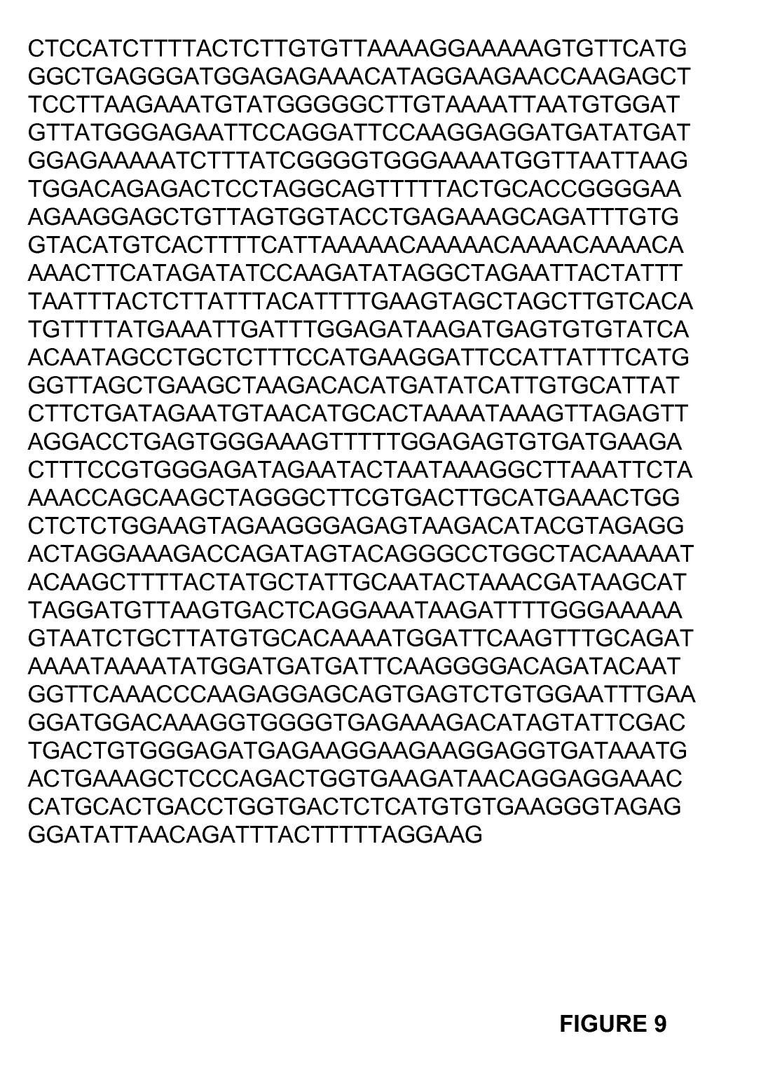

FIG. 9 shows:
The sequence of the human epsilon globin terminator sequence (SEQ ID NO:10).

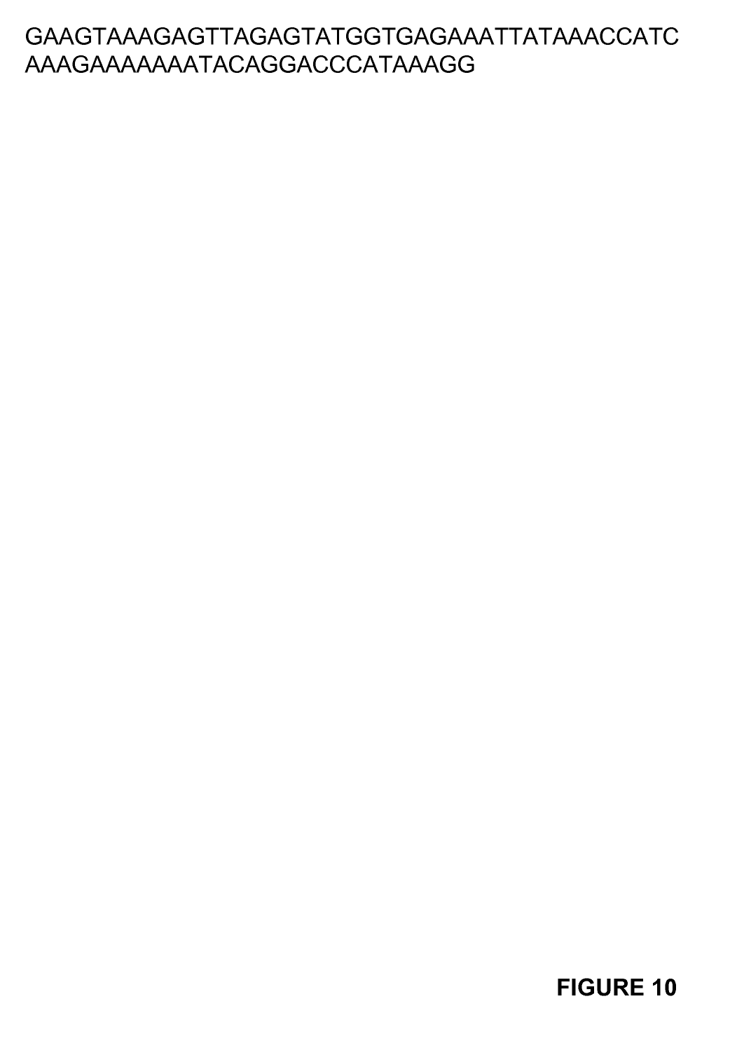

FIG. 10 shows:
The sequence of the mouse beta-major globin terminator sequence (SEQ ID NO:11).

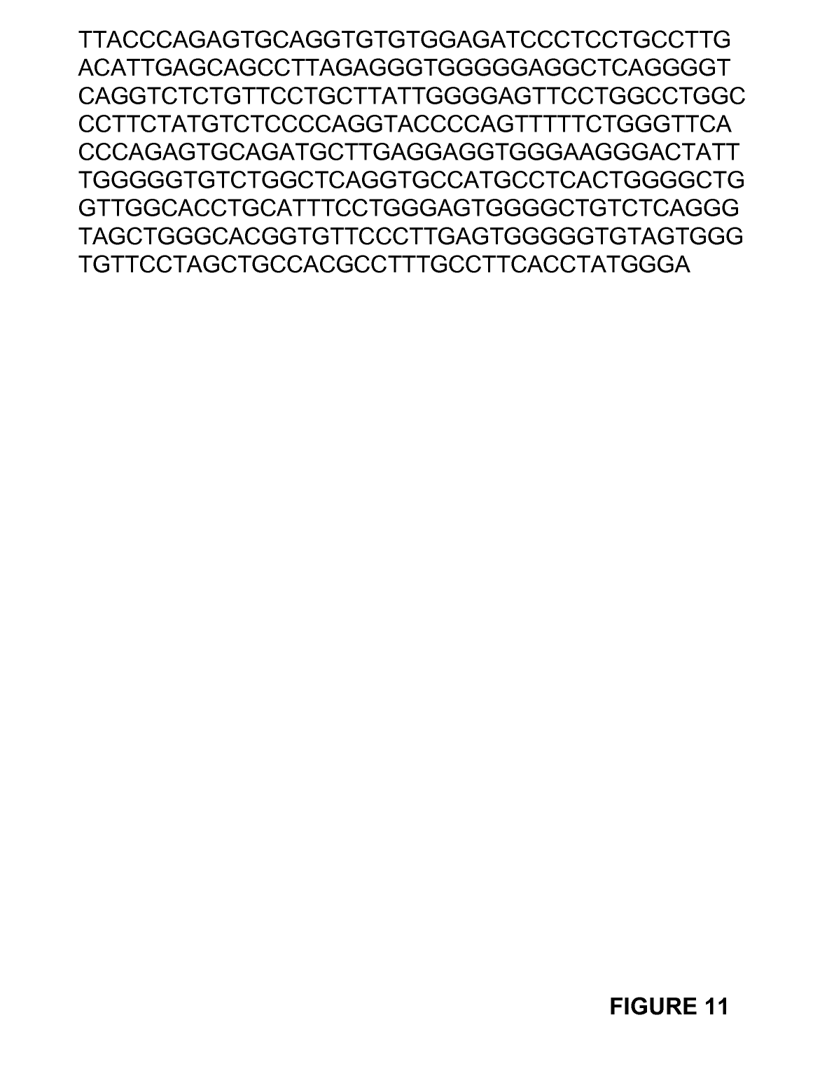

FIG. 11 shows:
The sequence of the human beta-actin terminator sequence (SEQ ID NO:12).

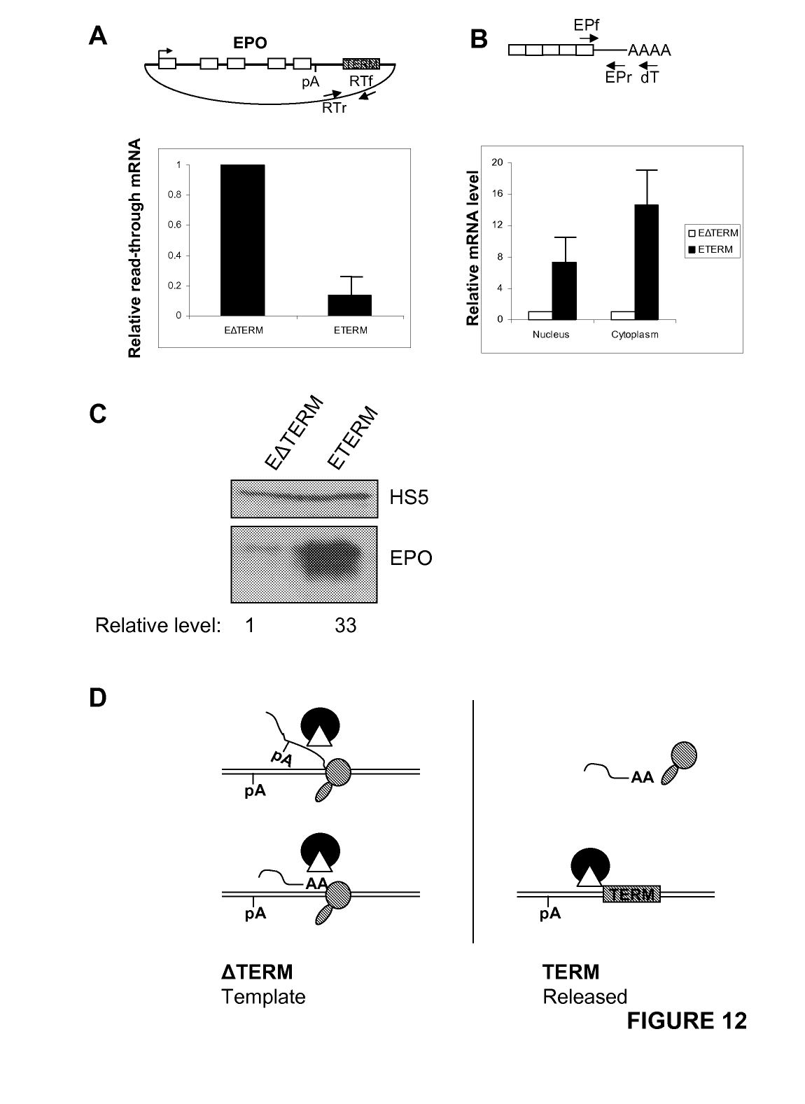

FIG. 12 shows:
A. Diagram shows ETERM with nomenclature the same as for βTERM (FIG. 1). Graph shows quantitation of read-through RNA in HeLa cells transfected with EΔTERM or ETERM as determined using real-time PCR. The value for EΔTERM was set at 1. Primers used for reverse transcription (RTr) and PCR (RTf/RTr) are shown on the diagram.
B. Real-time RT-PCR analysis of EPO mRNA levels in the nucleus and cytoplasm of HeLa cells transfected with EΔTERM or ETERM as well as VA. Values for EΔTERM were set at 1. Primers used for reverse transcription (dT) and PCR (EPf/EPr) are shown on the diagram.
C. Western blot analysis of EPO protein secreted from HeLa cells transfected with EΔTERM or ETERM. EPO (lower panel) and the HS5 control protein (upper panel) are indicated.
D. Model: In the absence of termination (left diagrams) pre-mRNA (top) and/or mRNA (bottom) is not released from the template region and is degraded by surveillance mechanisms (black pac-man). Efficient termination (right diagram) releases mRNA from transcription sites and away from the associated degradation machinery.

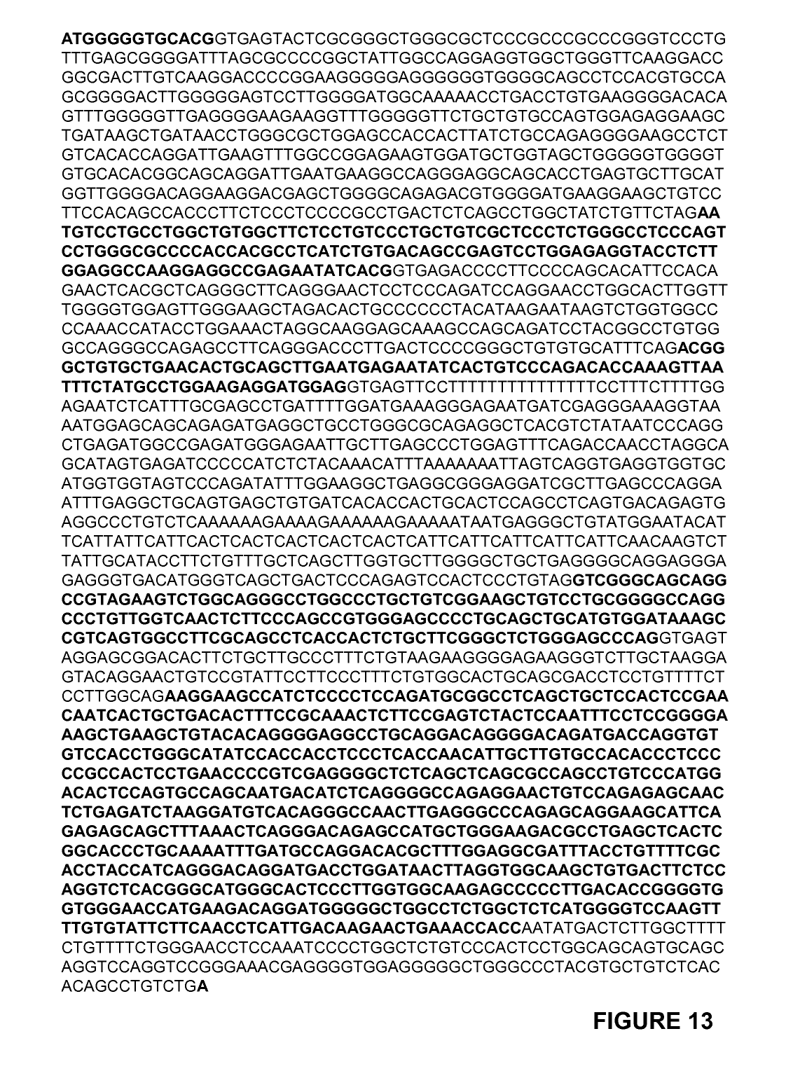

FIG. 13 shows:
The sequence of the human erythropoietin gene (SEQ ID NO:13).

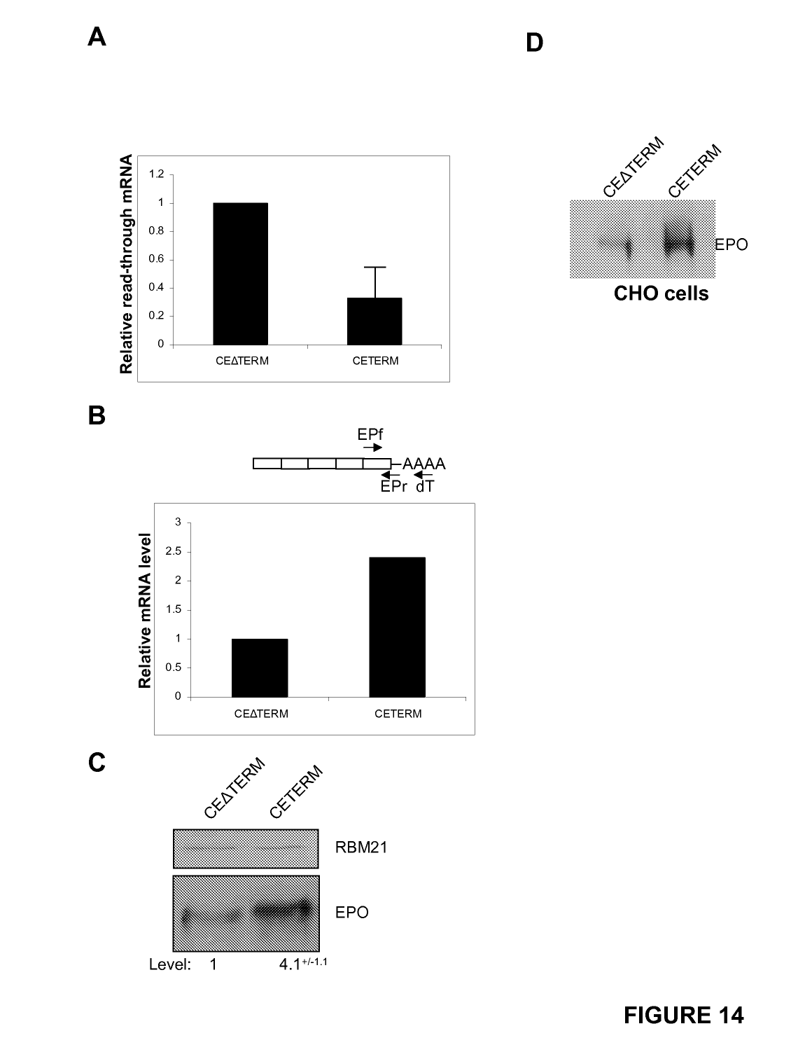

FIG. 14 shows:
A. RT-PCR quantitation of read-through RNA from CEΔTERM and CETERM. The ΔTERM sample are given a value of 1.
B. RT-PCR analysis of cytoplasmic EPO mRNA in HeLa cells transfected with CEΔTERM or CETERM. The ΔTERM sample are given a value of 1. Diagrams show primer for reverse transcription (dT) and PCR (e2f/e3r for β-globin and EPf/EPr for EPO).
C. Western blot analysis of EPO protein from HeLa cells transfected with CEΔTERM or CETERM. EPO and the RBM21 control protein are indicated and quantitated.
D. Western blot analysis of EPO protein from CHO cells transfected with CEΔTERM or CETERM.

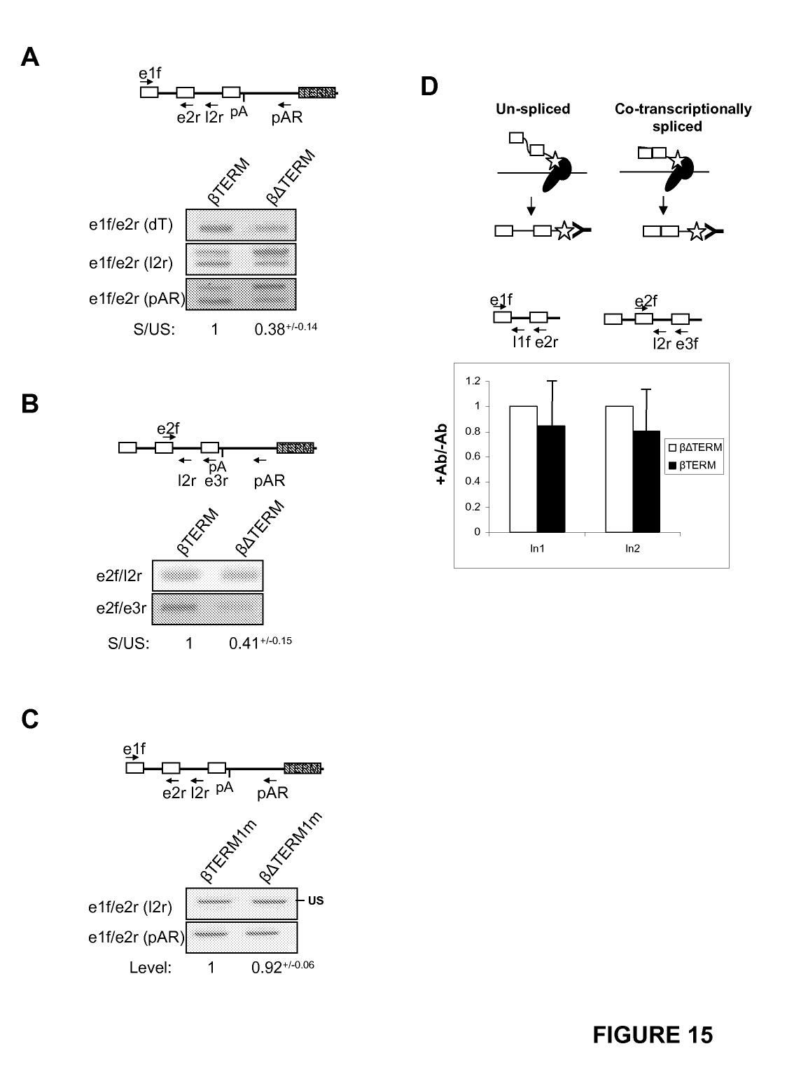

FIG. 15 shows:
A. RT-PCR analysis of intron 1 splicing in HeLa cells transfected with βTERM or βΔTERM. The diagram shows positions of the primers used in this experiment. Primer used for cDNA synthesis is indicated in brackets beside each panel with the PCR primer pair indicated to its left. Unspliced (US) and spliced (S) products are indicated. Real-time PCR quantitation of the ratio of spliced to unspliced (S/US) is shown.
B. RT-PCR analysis of intron 2 in HeLa cells transfected with βTERM or βΔTERM. Primers are indicated as in 5A. It should be noted that different cycle numbers were used for the separate PCR reactions. Real-time PCR quantitation of the ratio of spliced to unspliced (S/US) is shown.
C. RT-PCR analysis of pre-mRNA stability in HeLa cells transfected with βTERMml or βΔTERMml. Primers are indicated as in 5A. Real-time PCR quantitation is shown.
D. brUNRO analysis of co-transcriptional splicing. Top diagrams show the procedure, where immuno-precipitation of brU (star) detects co-transcriptional splicing (right) or introns that are not spliced during transcription (left). The lower diagrams show the primers used for reverse transcription (e2r and e3r) and PCR (e1f/I1r and e2f/I2r) to detect intron 1 and 2 respectively. Quantitation shows the signal (set at 1 for βΔTERM) obtained after subtracting the minus antibody control value.

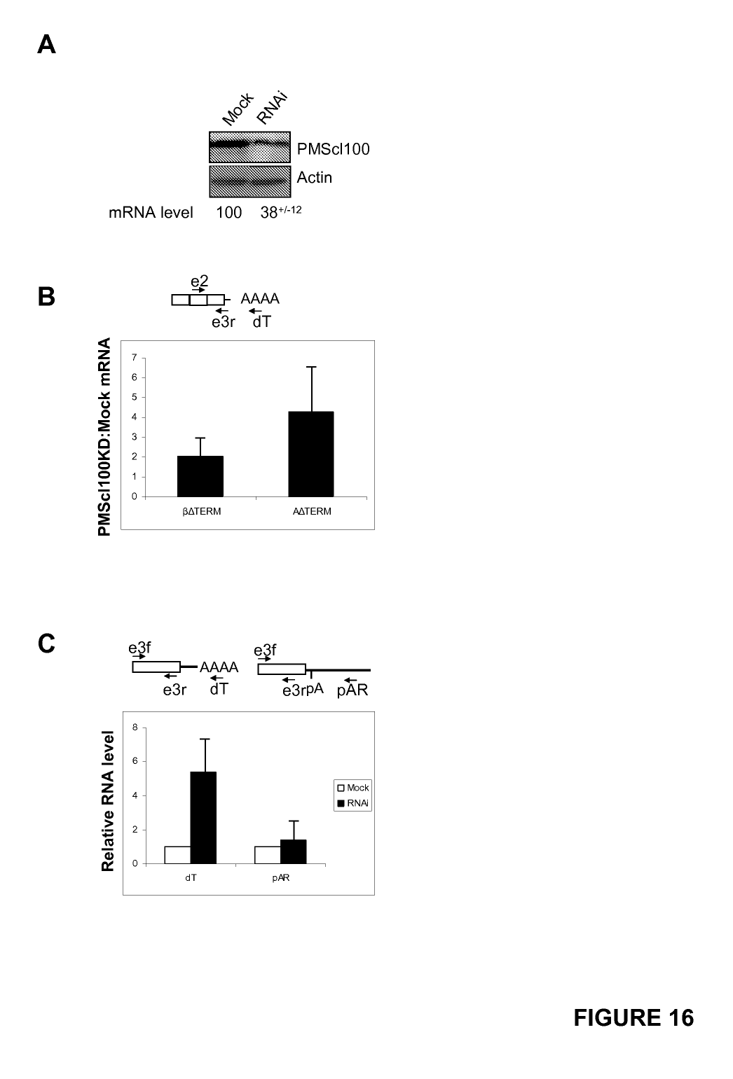

FIG. 16 shows:
A. Western blot analysis of PMScl100 and actin proteins from mock treated and PMScl100 depleted HeLa cells. Quantitation of PMScl100 mRNA is shown underneath.
B. Analysis of cytoplasmic β-globin mRNA in mock treated or PMScl100-depleted HeLa cells transfected with βΔTERM or AΔTERM. Graph shows the fold increase in cytoplasmic mRNA in PMScl100-depleted cells as compared to mock treated cells.
C. RT-PCR analysis of nuclear mRNA and pre-mRNA in HeLa cells transfected with AΔTERM. Diagrams show the target species (3′ end processed RNA on the left and RNA, not cleaved at the pA site, on the right). Primers used for reverse transcription (pAR and dT) and PCR (e3f/e3r) are also shown. Graph shows relative RNA levels (value for mock treated cells is 1).

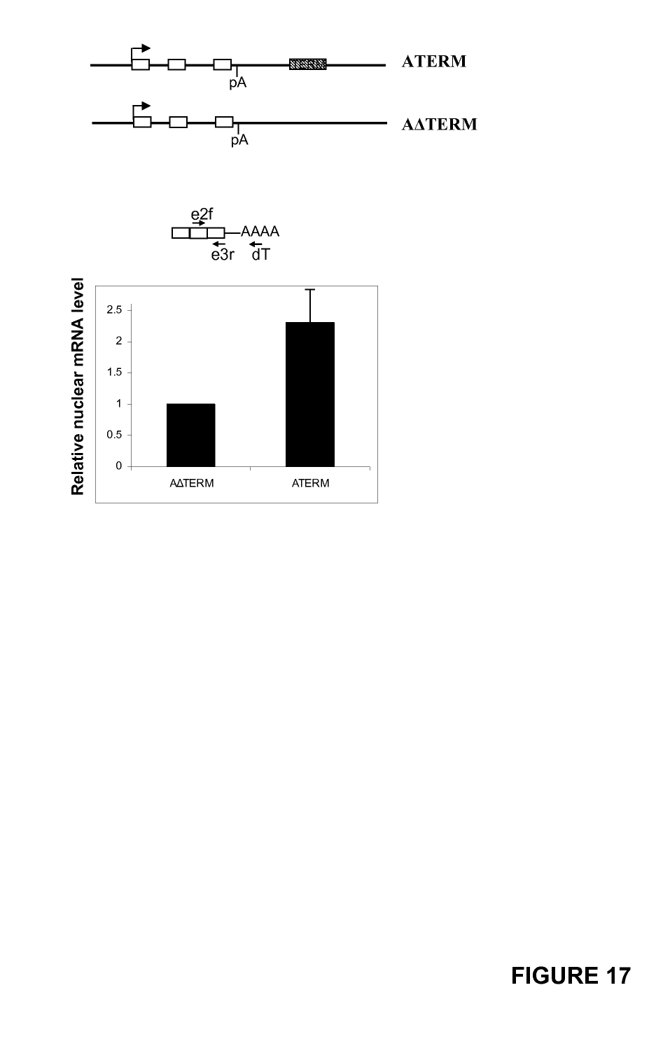

FIG. 17 shows:
Analysis of mRNA levels from linear templates.

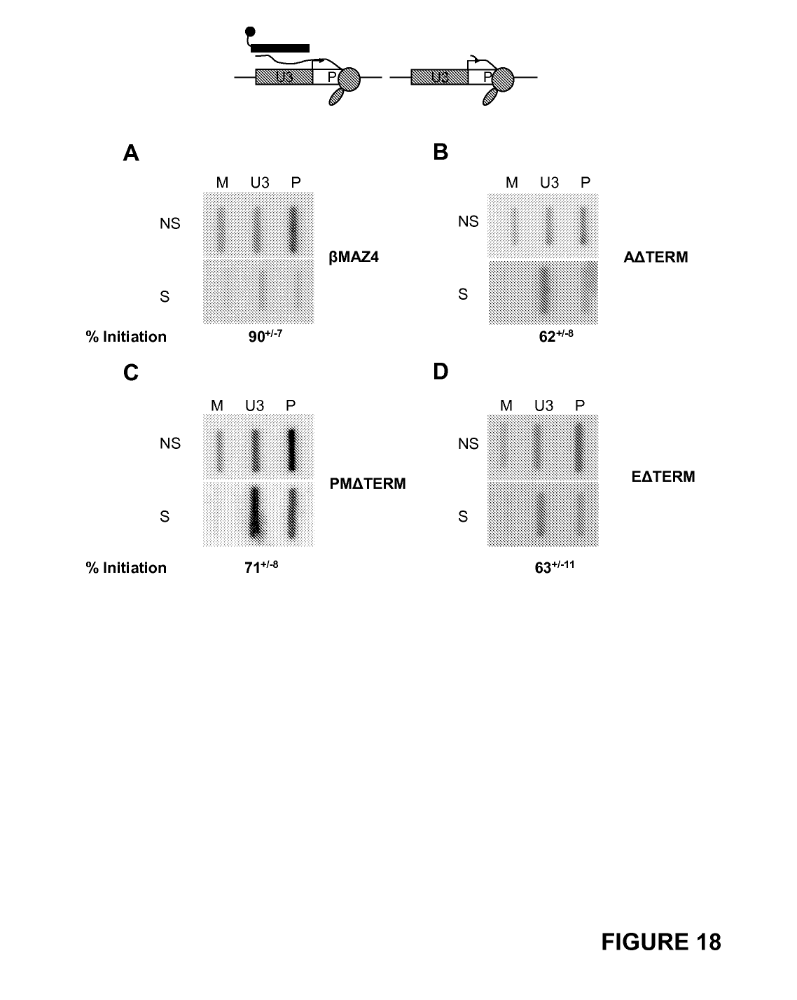

FIG. 18 shows:
Further analysis of transcriptional interference.

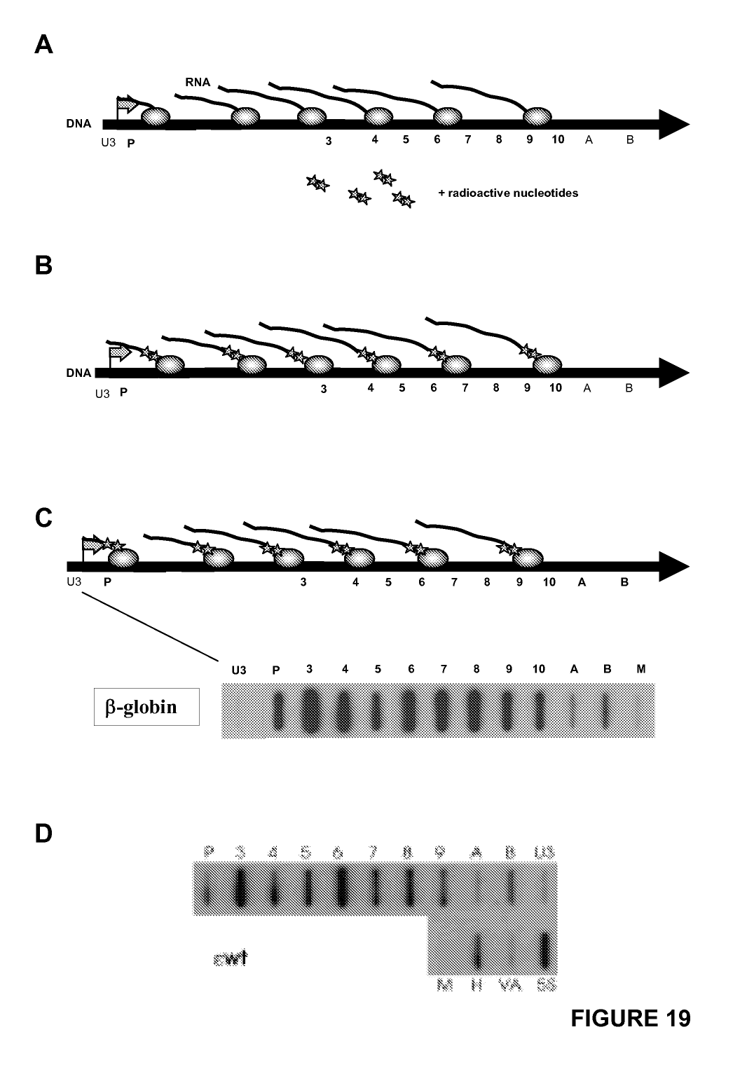

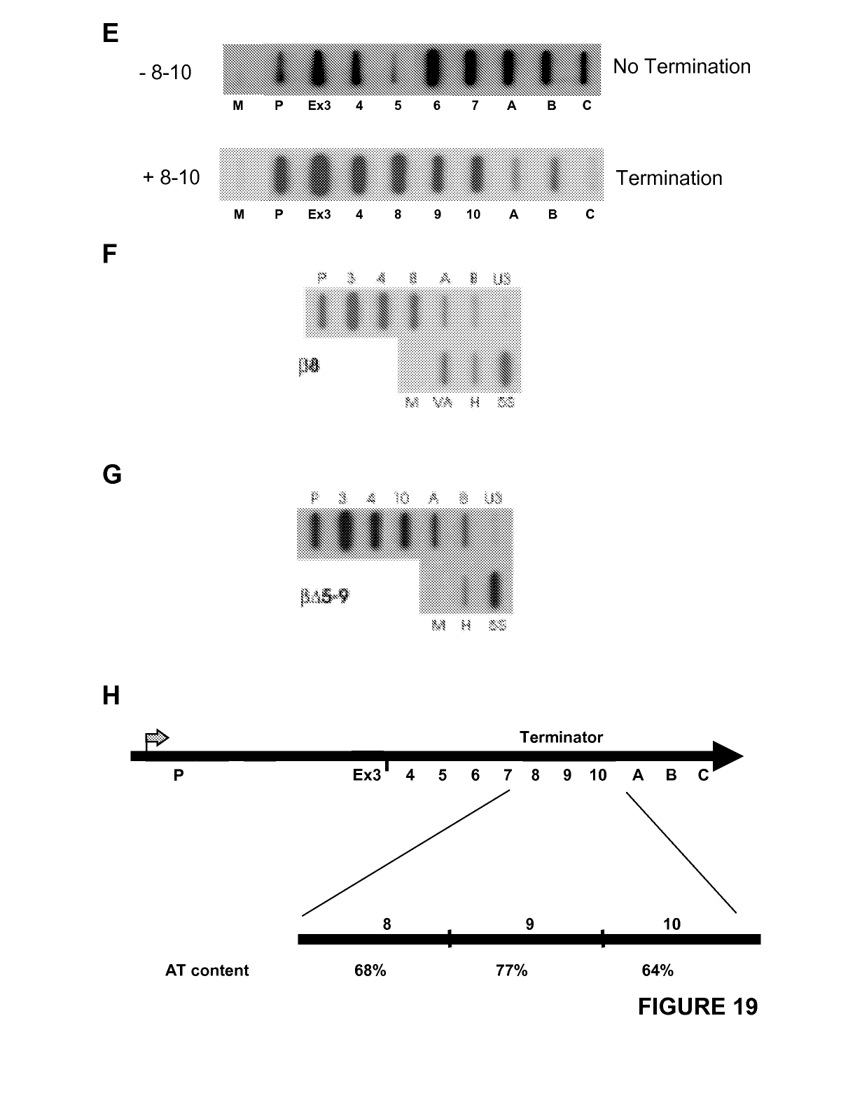

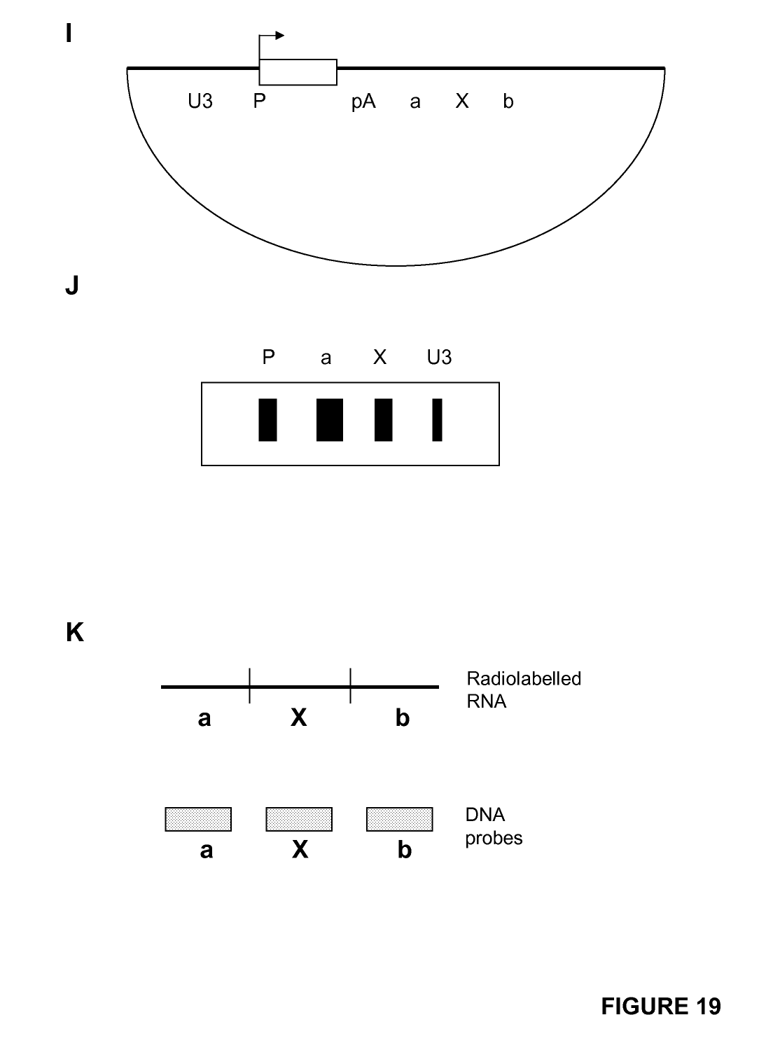

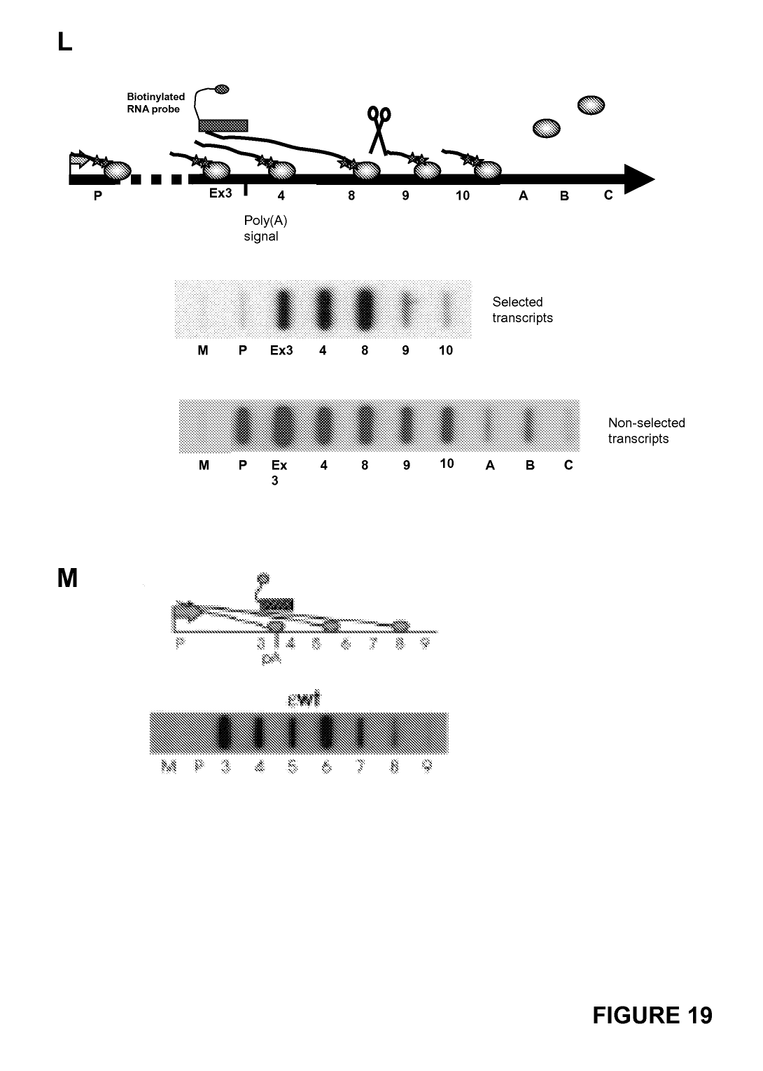

FIG. 19 shows:
A, B. Diagrammatic illustration of NRO analysis.
C. NRO analysis of the β-globin terminator region.
D. NRO analysis of the ε-globin terminator region.
E. NRO analysis of the β-globin terminator region (elements 8-10).
F. NRO analysis of the β-globin terminator region (element 8).
G. NRO analysis of the β-globin terminator region (element 10).
H. Diagrammatic illustration of the β-globin terminator region.
I. Diagrammatic illustration of a plasmid for use in NRO or hsNRO.
J. Diagrammatic illustration of labelled transcripts hybridised to probes which are complimentary in sequence to regions P, a, X and U3.
K. Diagrammatic illustration of labelled transcripts and DNA probes.
L. hsNRO analysis of the β-globin terminator region.
M. hsNRO analysis of the ε-globin terminator region.

FIG. 20 shows:
The mechanism of poly(A) signals and pause type terminators.

FIG. 21 shows:
The mechanism of CoTC type terminators.

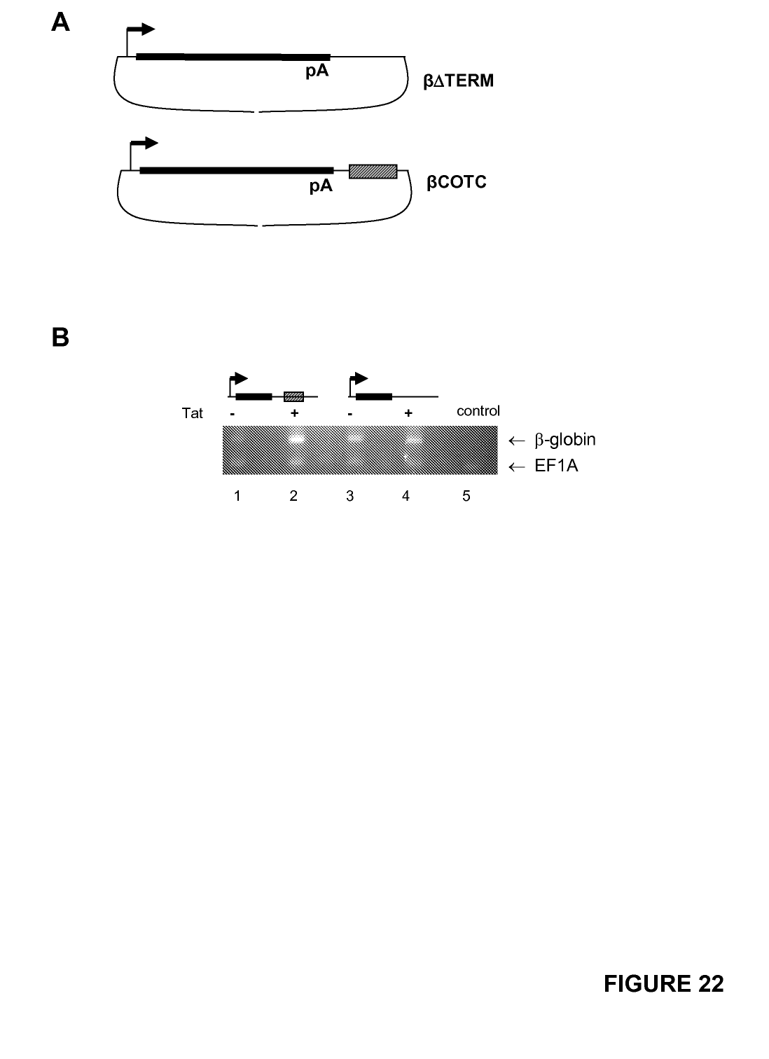

FIG. 22 shows:
A. Diagrammatic illustration of constructs used in experiments to show CoTC Terminator dependent gene expression enhancement in stably integrated genes.
B. RT-PCR analysis of mRNA from induced and non-induced cells incorporating the β-globin and β-globin+CoTC genes to detect and measure the level of integrated β-globin gene expression.

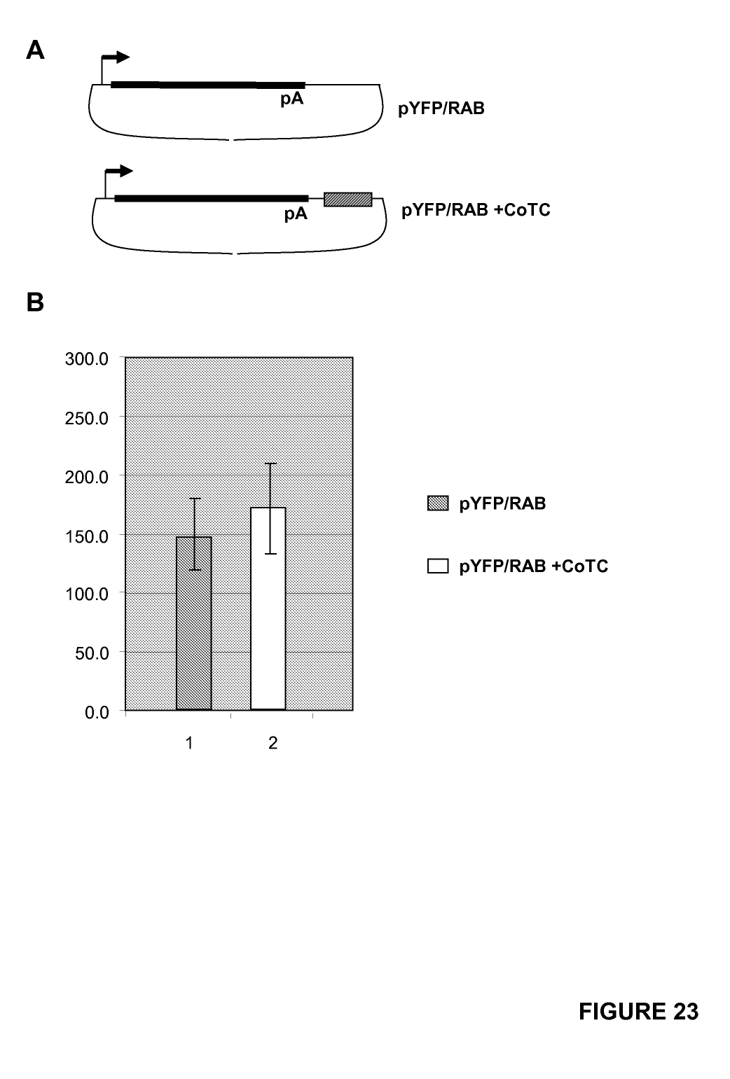

FIG. 23 shows:
A. Diagrammatic illustration of constructs used in experiments to show that the CoTC transcription termination element enhances protein expression in plants.
B. Analysis of YFP fluorescence in plant cells transfected with Agrobacterium YFP/RAB or Agrobacterium YFP/RAB+CoTC. Graph shows that YFP expression levels are higher in the cells transfected with Agrobacterium YFP/RAB+CoTC.

### DETAILED DESCRIPTION OF THE INVENTION

The invention is based on the demonstration that Pol II termination is required for optimal gene expression. In particular, it is shown herein that efficient termination enhances mRNA and protein levels. In the absence of a terminator sequence on a particular gene, a significant proportion of the mRNA transcripts produced from that gene are degraded. The terminator sequences disclosed herein, and other terminator sequences, cause the release of RNA polymerase II and its associated mRNA from sites of transcription and degradation and in so doing enhance mRNA processing. Thus, just as promoters initiate gene expression by binding Pol II, terminators enhance it by mediating its release.

The invention is particularly concerned with terminator elements that encode a section of RNA that is cut co-transcriptionally. Such terminator elements may encode a section of RNA that comprises a co-transcriptional cleavage (CoTC) substrate. Other such terminator elements may encode a section of RNA that comprises a ribozyme, optionally together with a pause type terminator sequence. mRNA produced from DNA containing such terminator sequences (that encode a section of RNA that is cut co-transcriptionally) is more efficiently translated, resulting in increased protein production. Accordingly the methods described herein enhance gene expression by enhancing both transcription and translation.

These findings may have wide ranging implications for in vivo and in vitro protein production, which may be enhanced by positioning terminator elements downstream (or 3′) of genes of interest. This is in contrast to current processes for expressing proteins from genes of interest which use DNA molecules comprising the gene of interest under the control of one or more promoter elements. The only sequence downstream of the gene which was considered to be of importance, until now, was the poly(A) signal. The invention is particularly valuable because any terminator sequence in accordance with the invention can be placed downstream of any gene to enhance its expression. Further, there are no particular requirements in relation to any intervening sequence between the poly(A) signal and the terminator sequence. In other words this intervening sequence does not need to correspond to the sequence naturally associated with either the selected gene of interest or the selected terminator sequence.

The terms “terminator sequence” and “terminator element” are used interchangeably herein to refer to a DNA sequence which is necessary for Pol II to terminate the transcription reaction. In other words, in the absence of the terminator sequence there would be no or inefficient termination of transcription in that Pol II would fail to release the DNA template and/or the RNA transcript. The most efficient terminator sequences are those which encode a section of RNA that is cut co-transcriptionally and it is these which are preferably used in accordance with the invention. Examples of such terminator elements include sequences encoding a CoTC substrate or a ribozyme. The terms “terminator element which encodes a section of RNA that comprises a CoTC substrate”, “terminator sequence which encodes a CoTC substrate”, “CoTC terminator sequence”, “CoTC type terminator sequence”, “CoTC terminator region” and the like are also used interchangeably herein.

CoTC Termination

The mechanism of CoTC terminators are shown in FIG. 21. Parts C/D shows the newly discovered mechanism of polymerase release and poly(A) site cleavage.

FIG. 21A. CoTranscriptional Cleavage of pre-mRNA. In the presence of a CoTC terminator element (CoTC in the lower diagram) the initial cleavage of the pre-mRNA is made at positions downstream of the poly(A) site within the RNA transcript encoded by the DNA CoTC element (scissors denote CoTC pre-mRNA cleavage sites).

FIG. 21B. Degradation of CoTC cleaved RNA transcripts. Following CoTC cleavage the polymerase continues transcribing and producing an RNA transcript. This transcript is degraded by 5′-3′ RNA exonucleases (circle with segment removed). During this time the pre-mRNA, not yet cleaved at the poly(A) site, remains attached to the transcribing polymerase.

FIG. 21C/D. When the downstream product of CoTC is completely degraded, polymerase along with the attached pre-mRNA, releases from the DNA template. After the polymerase is released from the DNA template the pre-mRNA is cleaved at the poly(A) site (scissors denote pre-mRNA cleavage at the poly(A) cleavage site). The cleaved pre-mRNA is further processed by the addition of a polyadenylate tail (AAAAAA in the diagram) to become the mature messenger RNA (mRNA) shown. Positioning of CoTC elements past the poly(A) site leads to an increase in the abundance of mature mRNA as indicated by the 4 mRNAs above the figure (as compared to the 1 or 2 mRNAs shown in FIG. 20). This increase in the level of mRNA exported to the cytoplasm consequently shows an increase in protein level. However there is not a linear relationship between the increase in the abundance of mRNA and protein level. The level of protein from each mRNA is significantly enhanced when the mRNA is derived from CoTC termination. This is shown by the large number of proteins in the figure. It is believed to be the release of polymerase before poly(A) site cleavage of the pre-mRNA that enhances the use (or recognition) of weak poly(A) sites such as that of the erythropoietin gene.

Thus, there are several differences between the two basic mechanisms of Pol II terminators—pause type terminators and CoTC type terminators:

1) The initial cleavage of the pre-mRNA is made at positions downstream of the poly(A) site within the RNA transcript encoded by the DNA CoTC element (these cleavage sites are referred to as the CoTC cleavage sites). For other termination mechanisms the initial cleavage is made at the poly(A) site.

2) Exonucleolytic degradation of the RNA downstream of the CoTC cleavage sites leads to release of the polymerase from the DNA template. At this stage, pre-mRNA, not yet cleaved at the poly(A) site, remains attached to the polymerase. In contrast, in the case of pause terminators, poly(A) cleavage site causes pre-mRNA release from the polymerase and subsequently exonucleolytic degradation of the RNA causes polymerase release from the DNA template.

3) In the case of CoTC type terminators, pre-mRNA is cleaved at the poly(A) site in association with the released polymerase. In the case of other terminators this process takes place on transcribing polymerase (that is polymerase engaged with the DNA template).

It is shown herein that CoTC termination enhances gene expression in a novel way. In particular, the level of mRNAs derived from the CoTC termination mechanism is higher than from previously described terminators because the pre-mRNA is processed away from the DNA template. This increase in mRNA occurs irrespective of the strength of the poly(A) signal. In contrast, other termination mechanisms enhance termination to a lesser degree when the poly(A) signal is weak.

Further, CoTC derived mRNAs are more efficiently translated into protein than mRNAs derived from other termination mechanisms. Thus, mRNA transcripts derived from a construct bearing a CoTC terminator lead to the production of more of the protein that they encode than identical mRNAs that derive from a construct that does not bear a CoTC terminator element.

Preferred terminator elements for use in accordance with the invention encode a section of RNA that is cut co-transcriptionally: that is it is cut whilst the polymerase continues to synthesise downstream parts of the same RNA chain and remains attached to the DNA template. In other words, such a section of an RNA chain is cut before the polymerase, which is synthesising that RNA chain, stops transcription and releases from the DNA template. Such co-transcriptional cleavage leads to release of the polymerase from the DNA before poly(A) site cleavage.

A preferred terminator element encodes a section of RNA that comprises a CoTC substrate, that is, it encodes a section of RNA that is cut co-transcriptionally and acts to enhance efficient termination of transcription. CoTC substrates are cut by an unknown mechanism. Few CoTC terminator regions are known and those that are differ extensively in sequence, which makes it difficult to perform genome-wide screens for such terminator elements or to identify them by sequence gazing. Such elements may be identified by analysis of an individual gene, or genes, of interest.

There is a direct correlation between the suitability of a section of an RNA molecule as a CoTC substrate and the extent to which it enhances transcriptional termination. In turn, there is a direct correlation between the suitability of a section of an RNA molecule as a CoTC substrate and the extent to which it enhances protein expression. Thus, the effectiveness of a sequence as a CoTC substrate may be determined by the efficiency with which it is able to terminate transcription.

Termination efficiency may be determined by a Nuclear Run On analysis. In accordance with the invention it is preferred that the terminator element is able to terminate the transcription reaction with a termination efficiency of 90% or more, preferably 95% or more, most preferably 99% to 100%.

An effective approach would involve cloning the 3′ flanking region of the gene of interest downstream of a model gene (such as β-globin) within a plasmid. The position of RNA polymerase II (Pol II) termination would then be analysed by Nuclear Run On analysis (NRO) which has previously been described in detail^{10, 11}. Following this, deletions within the 3′ flanking region would allow one to home in on the specific portion or portions necessary for termination.

Nuclear run on analysis is shown in FIG. 19. In this assay radioactive nucleotides (the building blocks of the RNA transcript, shown by stars) are added into an in vivo system in which active transcription of the gene of interest is occurring (FIG. 19A). The incorporation of the radioactive nucleotides into the RNA transcript, by elongating Pol II, results in the labelling of the RNA (FIG. 19B). Due to the fact that RNA transcripts hybridise very efficiently and stably to the DNA template from which they have been transcribed, the radiolabelled RNA transcripts can be used to map the position of active elongating Pol II on the DNA template. Radiolabelled transcripts are isolated and then hybridized to a panel of DNA probes, representing the gene regions under examination, that are fixed to a solid support. Exposure of X-ray film to the resulting RNA/DNA hybrids results in images such as that shown in FIG. 19C, which is essentially a transcription profile of the human β-globin gene.

The dark bands emanate from radioactive RNA molecules hybridized to their cognate DNA sequence. Thus reading FIG. 19C from left to right it can be seen that there is no signal over probe U3, which is positioned immediately upstream of the promoter (the start site of transcription, probe P). There are strong signals between P and 10 (variation in signal strength over probes P-10 is due to variations in the length and strength of each RNA/DNA hybrid). The nuclear run on data shows that transcription begins at P (promoter) and continues up to probe 10. The very low signals downstream of 10, over probes A and B are due to non specific hybridization to cellular RNA transcripts and are very close to the background level of non-specific RNA/DNA hybridization, indicated by probe M. These signals are therefore considered to be at or near zero. The absence of signal beyond probe 10 indicates that transcription has terminated. The β-globin gene has the most efficient transcription termination profile that we have seen and we consider that it operates with 100% efficiency. That is, 100% of polymerases that begin transcription at the promoter will terminate by the time they have passed probe 10.

This situation contrasts with that of the human ε-globin gene when subjected to the same analysis, as shown in FIG. 19D. Hybridisation signals over probes A, B and U3 are relatively higher (compare probes P and U3). The ε-globin gene contains reasonable termination signals but they do not demonstrate the very high efficiency of the β-globin gene.

This assay may be used to examine the role of DNA sequences in transcription termination. For example, variants of the β-globin gene, including different sections of the DNA sequence located downstream of the pA signal (regions 4-10 in FIG. 19) were constructed and analysed by nuclear run on analysis. It was found that transcription termination was mediated by the DNA sequences in region 8, 9 and 10 (FIG. 19E). Further experiments were conducted to determine which DNA sequences within the 8-10 region were important in termination. It was found that each section, 8, 9 or 10 could mediate efficient termination as measured by Nuclear Run On and other transcription assays. FIGS. 19F and G show that regions 8 and 10, respectively, are able to mediate efficient termination independently.

The fact that sub-sections of the β-globin terminator are sufficient to direct 100% transcriptional termination indicates that it is an extraordinarily strong transcription control element (compare signals over probes U3 and P, i.e. the strength of hybridisation signal from polymerases positioned before the terminator region with the strength of hybridisation signals from polymerases positioned after the terminator region). This makes sense when one considers the fact that there are only two copies of the human β-globin gene in erythrocytes. These two copies are extremely active producing large amounts of β-globin messenger RNA in the adult human. Transcription has to be very efficient in this system. It is possible, when considering this biological background, that the β-globin terminator is the most efficient terminator and we consider that the complete 850 bp terminator or ˜300 bp sub-sections of it demonstrate 100% termination efficiency. The characteristics of the termination region are shown in FIG. 19H.

In summary, nuclear run on analysis is employed to measure the activity and position of RNA polymerase. The nascent RNA transcripts are labelled as they are being made by the endogenous polymerase. Thus only nascent transcripts that have incorporated the label are visualised. The position of the polymerase is inferred by hybridising the resulting labelled RNA transcripts to complimentary nucleic acid of known sequence. Nuclear run on analysis is thus an unequivocal method for measuring the extent of active transcription on a certain DNA sequence. It is therefore also an unequivocal method for determining which DNA sequences regulate or influence the transcription processes such as transcription termination. The ability of a DNA sequence to promote transcriptional termination is measured by analysing the density of polymerases that transcribe beyond it.

This is illustrated in FIG. 19I which shows a plasmid DNA molecule comprising a promoter (P), followed downstream by a gene (box), a polyA signal (pA), and regions a, X (a potential terminator element) and b. Region U3 is downstream of region b and upstream of the promoter. Upon transcription, labelled transcripts are produced. The labelled transcripts are hybridised to probes which are complimentary in sequence to regions P, a, X and U3, as illustrated diagrammatically in FIG. 19J.

Termination efficiency is determined by comparing the relative strengths of the hybridisation signals over probes P and U3. The termination efficiency is defined as compared to when X is represented by the human β-globin terminator (SEQ ID NO:1), which is considered to terminate transcription with 100% efficiency. This is based on the relative levels of P and U3 and with the β-globin terminator the P/U3 ratio reflects 100% termination.

If cloning 3′ flanking regions does not reveal a terminator element then it could be that one is not present or that termination occurs beyond the region analysed. In the case of the latter, one could clone even more 3′ flanking region and repeat the analysis described in the paragraph above. Alternatively, northern blotting/reverse transcription PCR and Pol II-specific chromatin immunoprecipitation could be used to identify the extent of transcription on the endogenous gene and the segment of DNA over which Pol II terminates could be isolated and analysed as described in the paragraph above.

The effectiveness of a terminator element may also be determined by directly analysing the co-transcriptional cleavage activity. Transcripts from CoTC terminators (i.e. CoTC substrates) and from other ‘artificial’ CoTC terminators (e.g. that encode ribozymes) are co-transcriptionally cleaved and this activity can be identified at a particular sequence experimentally using hybrid selection nuclear run on analysis (hsNRO), conducted as detailed in Dye and Proudfoot, 1999¹⁰ and 2001¹. This assay measures the continuity of nascent RNA transcripts and therefore allows identification of CoTC substrates or other RNA sections that are cut co-transcriptionally. The finding that β- and ε-globin terminator region transcripts are CoTC substrates led to the connection being made between CoTC and transcriptional termination.

Initially, radio-labelled nucleotides are incorporated into nascent transcripts using nuclear run on (NRO) analysis as described¹¹. The labelled transcripts are then hybridised to an anti-sense biotinylated RNA probe¹⁰. Hybrids are then selected with streptavidin-coated magnetic beads. Selected transcripts are then hydrolysed and hybridised to anti-sense nucleic acid probes spanning the terminator region as described^{1, 10}. If the terminator is a CoTC element, one will be unable to select all of the transcripts that span the region as cleavage will render them discontinuous with the upstream region to which the biotinylated probe hybridises.

This is illustrated in FIG. 19I which shows a plasmid DNA molecule comprising a promoter (P), followed downstream by a gene (box), a polyA signal (pA), and regions a, X (a potential terminator element) and b. Upon transcription, labelled transcripts are produced. Sections a, X and b of the labelled transcripts are shown in FIG. 19K.

To measure CoTC activity in the radiolabelled RNA, the transcript is selected away from the RNA pool by an antisense probe which is complementary to region a of the RNA. The selected RNA transcripts are then hybridised to the DNA probes a, X and b. The efficiency of CoTC in region X is then determined by the strength of hybridisation signal over probe b, which lies downstream of the CoTC substrate region.

If region X under analysis is co-transcriptionally cleaved with 100% efficiency, then no b region radiolabelled RNA will be selected. In this instance, region b no longer constitutes part of the same molecule as region a—it is not linked to it because it has been disconnected by the cutting of the RNA chain in the CoTC region. If, on the other hand, there is no CoTC activity in the putative CoTC region X, then there will be a strong hybridisation signal over the DNA probe b.

In order to quantitate the efficiency of the CoTC substrate, each sequence analysed may be compared to a sequence with 100% CoTC activity, that is a sequence that is cleaved to the extent that prohibits the selection of any downstream RNA transcripts that, if it were not for CoTC activity, would be continuous with it. Elements of the human β-globin gene terminator region (SEQ ID NOS: 2, 3 and 4) have been shown to be the most efficient CoTC substrates, in terms of their short length and the degree to which they are cut. When these elements are substituted in region X as described above and subjected to hsNRO analysis, then no radiolabelled transcripts positioned downstream of region X are retrieved by hybrid selection, i.e. no radioactive signal over b would be detected following hybrid selection. It is considered that these sequences are co-transcriptionally cleaved with 100% efficiency. Further, the efficiency of these human β-globin fragments as terminators, analysed by NRO, corresponds directly to their degree of cutting in the hsNRO analysis.

Less efficient CoTC substrates will show correspondingly higher hybridisation signals over region b in the hsNRO analysis. In accordance with the invention it is preferred that the terminator element is able to cleave co-transcriptionally with an efficiency of 50% or more, preferably 75% or more, 80% or more, 90% or more, 95% or more, or most preferably 99% to 100%. The efficiency of co-transcription cleavage is defined by the above-described hsNRO analysis, as compared to a corresponding analysis of SEQ ID NO:2 (element 8 of the human β-globin terminator) which is considered to terminate transcription with 100% efficiency, i.e. no radioactive signal over region b would be detected following hybrid selection.

Control experiments may be conducted to establish the range of CoTC efficiency and thus the efficiency of a certain CoTC substrate. Referring again to FIG. 19, a control for no CoTC can be carried out by omitting region X (the potential CoTC substrate). Where there is no CoTC substrate between probes a and b then a hybrid selection experiment will result in a strong hybridisation signal over probe b, representing 0% CoTC. The establishment of a range of CoTC efficiency from 0% to 100% allows the CoTC efficiency of any RNA molecule to be determined and expressed in %.

To be sure that the terminator is a CoTC terminator, the upstream pA signal should be mutated and the experiment repeated. In this case, no transcripts extending beyond the terminator should be selected. If the terminator is not a CoTC element, transcripts beyond it will be selected with this technique.

Because of the extreme instability of nascent RNA transcripts care must be taken when conducting hsNRO experiments. Control experiments that must be undertaken in order to correctly measure and assign CoTC activity are detailed in the publications cited above^{1, 10}.

This technique (hsNRO) showed that transcripts of the termination regions were cleaved as soon as they were synthesized by RNA Pol II, as shown in the FIG. 19L in connection with the β-globin terminator region (elements 8 to 10). This data shows that nascent transcripts of the termination region are cleaved as soon as they are transcribed. The data above also show that transcripts are cleaved at the end of region 8/beginning of region 9. The same analysis was applied to sub sections 9 and 10 of the human β-globin termination region and it was found that they also mediate the transcript cleavage event (Co-Transcriptional Cleavage or CoTC). The terminator region transcripts, 8, 9 and 10 are substrates of this activity. There is a clear correspondence between the efficacy of a DNA element as a transcription terminator and its suitability as a CoTC substrate.

This is supported when analyzing CoTC of the transcripts of weak termination elements such as the human ε-globin gene terminator (FIG. 19M). Here selected transcripts extend throughout the termination region with no clear cut off point. Thus transcripts of the weak ε-globin termination sequences are not good CoTC substrates. This correspondence between termination and CoTC activity has been shown for the mouse serum albumen⁵ and the human γ and α-globin genes¹⁵.

An alternative, but currently less accurate method for identifying a CoTC substrate is by selection of chromatin-associated and released nuclear RNA. Nuclear RNA is fractionated into chromatin-associated (C) and released (R) fractions as described^{2, 13}. Pre-mRNA (from the gene containing the suspected CoTC terminator) that has yet to be cleaved at the poly(A) site is then analysed by RT-PCR. If the terminator is a CoTC terminator, a large fraction (at least 40%) of this pre-mRNA will be in the R fraction. The majority of transcripts (at least 70%) from non-CoTC terminators that are not cleaved at the poly(A) site will be restricted to the C fraction.

Another preferred terminator element encodes a section of RNA that comprises a ribozyme. Such terminator elements are also cleaved co-transcriptionally and promote efficient termination of the transcription reaction, leading to enhanced levels of expressed mRNA and protein. It has been shown that an efficient hammerhead ribozyme (RZ) cleaves itself co-transcriptionally and that when positioned upstream of MAZ transcription pause sites operates to enhance transcriptional termination as CoTC substrates do^{2, 23}. The RZ/MAZ combination leads to release of polymerase from the DNA template as do CoTC terminators. Therefore the ribozyme/pause site combination is an example of this type of terminator. A specific example is shown in SEQ. ID NO:45.

However, such terminators may comprise any ribozyme, including natural and synthetic ribozymes. Examples include Peptidyl transferase 23S rRNA, RNase P, Group I and Group II introns, GIR1 branching ribozyme, Leadzyme, Hairpin ribozyme, Hammerhead ribozyme, HDV ribozyme, Mammalian CPEB3 ribozyme, VS ribozyme and glmS ribozyme. Preferred is an autocatalytic hammerhead ribozyme, for example having the following sequence:

(SEQ ID NO: 47)
CCTGTCACCGGATGTGTTTTCCGGTCTGATGAGTCCGTGAGGACGAAAC
AGG

The terminator element encoding a ribozyme may also comprise a terminator sequence, such as a pause type. The pause type terminator sequence is preferably positioned downstream of the ribozyme. Thus, the terminator element may comprise a ribozyme sequence followed by one or more pause type terminator sequences. For example, the terminator element may comprise an autocatalytic hammerhead ribozyme (such as SEQ ID NO:47) followed by one or more pause type terminator sequences, such as MAZ (SEQ ID NO:6) or MAZ4 (SEQ ID NO:46).

In a specific example this sequence is inserted downstream of the poly(A) cleavage site (for example 90 nucleotides downstream of the poly(A) cleavage site). A further 120 nucleotides downstream of this sequence, a pause terminator comprising 4 MAZ sites is inserted (4× GGGGGAGGGGG (SEQ ID NO:6) or GGCCGCGCCGTCGACCTGGCCTTGGGGGAGGGGGAGGCCAGAATGAGAGC TCCTGGCCTTGGGGGAGGGGGAGGCCAGAATGACTCGACCTGGCCTTGGGG GAGGGGGAGGCCAGAATGAGAGCTCCTGGCCTTGGGGGAGGGGGAGGCCA GAATGACTCGAGGAATTCCCATGCA (SEQ ID NO: 46)).

The entire sequence of this terminator element is:

(SEQ ID NO: 45)
CCTGTCACCGGATGTGTTTTCCGGTCTGATGAGTCCGTGAGGACGAAAC
AGGCCTTGGGAAAATACACTATATCTTAAACTCCATGAAAGAAGGTGAG
GCTGCAAACAGCTAATGCACATTGGCAACAGCCCTGATGCCTATGCCTT
ATTCATCCCTCAGAAAAGGATTCAAGGGCCGCGCCGTCGACCTGGCCTT
GGGGGAGGGGGAGGCCAGAATGAGAGCTCCTGGCCTTGGGGGAGGGGGA
GGCCAGAATGACTCGACCTGGCCTTGGGGGAGGGGGAGGCCAGAATGAG
AGCTCCTGGCCTTGGGGGAGGGGGAGGCCAGAATGACTCGAGGAATTCC
CATGCA.

Terminator elements that encode a section of RNA that comprises a ribozyme may be analysed as described above in terms of their ability to terminate the transcription reaction (by NRO analysis) and their ability to cleave the RNA transcript co-transcriptionally (by hsNRO analysis).

The terminator element may be from about 20 to 2000 nucleotides in length, or 100 to 1500 nucleotides in length, or from about 250 to 1200 nucleotides, or from about 400 to 900 nucleotides. Preferably the terminator element may be about 250 or 300 nucleotides or longer. The consideration of length of the terminator element is very important for practical reasons, cloning etc and because rapid termination is preferable for enhancing mRNA production and stability. Rapid termination and polymerase release means that the pre-mRNA is quickly removed from the vicinity where competing process such as pre-mRNA degradation are taking place.

Preferably the terminator element is AU rich in that the RNA encoded by this element contains at least about 60% A and/or U residues, more preferably at least about 65% A and/or U residues, more preferably at least about 70% or 75% A and/or U residues.

Suitable terminator sequences, that encode a section of RNA that is cut co-transcriptionally, for use in the present invention include:
Human β-globin terminator sequence (GenBank sequence accession number U01317, nucleotides 64568-65421) (FIG. 5a) (SEQ ID NO:1)
Element 8 of the human β-globin terminator sequence (GenBank sequence accession number U01317, nucleotides 64568-64938) (FIG. 5b) (SEQ ID NO:2)
Element 9 of the human β-globin terminator sequence (GenBank sequence accession number U01317, nucleotides 64939-65126) (FIG. 5c) (SEQ ID NO:3)
Element 10 of the human β-globin terminator sequence (GenBank sequence accession number U01317, nucleotides 65127-65421) (FIG. 5d) (SEQ ID NO:4)
Mouse albumin terminator sequence (FIG. 6) (SEQ ID NO:5)
Human gamma A globin terminator sequence (FIG. 7) (SEQ ID NO:8)
Human gamma G globin terminator sequence (FIG. 8) (SEQ ID NO:9)
Human epsilon globin terminator sequence (FIG. 9) (SEQ ID NO:10)
RZMAZ4 sequence (SEQ ID NO:45).

In preferred embodiments, the terminator element comprises the human β-globin terminator region as set out in SEQ ID NO:1 or SEQ ID NO:45 or fragments or variants thereof. A fragment is defined herein as a sequence which is sufficient to terminate the transcription reaction, in that a termination efficiency of 90% or more, preferably 95% or more, most preferably 99% to 100% is achieved, as determined by Nuclear Run On analysis, as discussed above. Alternatively or additionally, a fragment may also be defined as a sequence which has CoTC activity as determined by hsNRO, in that co-transcriptional cleavage occurs with an efficiency of 50% or more, preferably 75% or more, 80% or more, 90% or more, 95% or more, or most preferably 99% to 100%, as determined by hsNRO, as discussed above. A variant is defined herein as being about 90% or more identical to the specified sequence, preferably about 95% or more identical and most preferably about 98% or more, about 99% or more or about 100% identical to the specified sequence.

In other preferred embodiments the terminator element comprises one or more of elements 8, 9 and 10 of the human β-globin terminator sequence as set out in SEQ ID NO:2, SEQ ID NO:3 and SEQ ID NO:4, respectively, or a variant thereof.

As well as the specific terminator sequences disclosed herein, one or more elements of these sequences which is sufficient to terminate transcription, by causing Pol II to release the DNA template and/or the RNA transcript, may be used. For example, elements of around 250 bp of the human β-globin terminator sequence are sufficient to terminate transcription, as shown in SEQ ID NOS: 2 to 4. Other such elements could be identified by the skilled person in the manner discussed above.

The expression of any protein-coding gene may be enhanced in accordance with the method of the claimed invention. For example, it may be desirable that the gene of interest encodes a protein comprising erythropoietin, interferon protein, insulin, a growth hormone, a clotting factor, a viral antigen, an antibody or an enzyme. In general the enhancers according to the invention can be used with advantage in any case where the value of an organism or cell line to commerce, including agriculture, is determined by the level of expression of one of its natural genes or of an artificially introduced gene. For example:

(i) In the manufacture of commercially useful proteins, such as erythropoietin, in genetically engineered organisms or cell lines.

(ii) In fermentation where the rate of production of a product of intermediary metabolism catalysed by enzymes can be enhanced by increasing the rate of synthesis of a particular enzyme.

(iii) In the expression of a partial clone of a gene, for example where it is desired to raise antibodies to the product of expression of the partial clone.

(iv) In the genetic engineering of plants where a property such as herbicide resistance is installed by artificial modification of the intermediary metabolism of the plant.

(v) In gene therapy of a patient where it is desired to increase the expression of a particular protein which is not produced naturally in sufficient levels or in a functional form by the patient.

In accordance with the claimed invention any terminator sequence can be used to enhance the expression of any gene of interest. Although the terminator sequence that is selected may be naturally associated with the gene that it is desired to express (e.g. enhancing expression of the β-globin gene using the β-globin terminator sequence, as in Example 1 below), this is not necessary.

In most cases it is not advantageous to include multiple terminator sequences because the terminator sequence will terminate transcription efficiently by itself. For example, the various human β-globin terminator regions terminate transcription with up to 100% efficiency. However, there are instances when it may be advantageous to include multiple terminator sequences in order to ensure efficient transcription termination, for example, with respect to the MAZ and ZAM sequences, the more repeats of these sequences, the more efficient termination is.

Once the gene to be expressed and the terminator sequence have been selected, the terminator sequence should be cloned downstream of the gene of interest by any technique (such as restriction digestion/PCR/ligation), as will be well known to the skilled person.

The gene will, of course, also have an associated promoter element upstream, as well as a poly(A) signal sequence downstream to provide a poly(A) addition site. Any promoter sequence may be used in accordance with the invention, including for example the CMV promoter, SV40 promoter, TK promoter, RSV promoter, Adenovirus Major Late promoter or HIV promoter. Often the poly(A) signal will have the consensus sequence AATAAA; however the skilled person will recognise that other sequences can perform the same function, to varying degrees of efficiency, and will understand that these sequences are also referred to as poly(A) signal sequences. Poly(A) signals that can be used in accordance with the invention include AATAAA, ATTAAA, the MSA poly-adenylation signal, the EPO poly-adenylation signal and the PMScl100 poly-adenylation signal. An important aspect of the CoTC terminator is that it enhances gene expression when positioned downstream of any poly(A) signal. Notably, enhanced gene expression is observed even when a ‘weak’ or inefficient poly(A) signal (such as the EPO poly-adenylation signal or the MSA poly-adenylation signal) is used. Further, relative enhancement is higher when a weak poly(A) site is present, although even higher absolute levels of protein would be produced if the weak poly(A) site was replaced with a strong one (in the presence of a CoTC terminator).

In mammals, transcriptional termination has been shown to occur at varying distances downstream of the poly(A) signal (between approximately 150 base pairs (bp) and 4000 bp). Accordingly it is preferred that the terminator sequence is located from 0 to 5000 bp downstream of the poly(A) signal, preferably from 150 to 4000 bp downstream of the poly(A) signal, more preferably from 200 to 3000 bp, 200 to 2000 bp, 200 to 1500 bp, or most preferably around 300 bp downstream of the poly(A) signal. There are no specific requirements in relation to the sequence located between the poly(A) signal and the terminator sequence.

There is no requirement for the terminator sequence to be in frame with any of the upstream sequences.

The DNA molecule having a sequence which comprises in a 5′ to 3′ direction (i) one or more promoter elements, (ii) the gene of interest, and (iii) a poly-adenylation signal, and (iv) a terminator element, is usually in the form of an expression vector. An expression vector is any vector capable of expressing those DNA sequences contained therein which are operably linked to other sequences capable of effecting their expression. An example of an expression vector is a plasmid.

Once the DNA molecule has been produced, it can be amplified and used in a suitable expression system, as is well known to the skilled person. The expression vector may be introduced directly into a host cell stably or transiently where the DNA sequence is expressed by transcription and translation. In the case of stable expression the vector must be replicable in the host either as an episome or as an integral part of the chromosomal DNA.

Any expression system may be used, including cell or tissue cultures and cell-free systems. Suitable expression systems include mammalian cells, insect cells, plant cells, bacterial cells and yeast cells. Preferred expression systems are human and mammalian tissues and cell lines, for example HeLa cells, 293T cells, CHO cells, HEP G2 cells. Expression of protein from the gene of interest can be induced in the usual way and this will depend on the type of promoter element(s) used.

The enhancers according to the invention can be used in any situation where it is desired to enhance the expression of a gene. However it should be noted that the effect of the enhancers is most marked in cases where termination of transcription is fully or partially inefficient, for example in the case where the poly(A) signal is inefficient. Thus the addition of an enhancer according to the invention will assist in efficient termination of transcription. However, the effect of the enhancer according to the invention is still present in enhancing the expression of genes which do contain an efficient poly(A) signal in their sequence.

Yields of mRNA and protein can also be quantified by numerous techniques known to the skilled person, including real-time PCR, northern blot, RNAse protection and S1 nuclease analysis for mRNA yields, and Western blot for protein yields.

When the term “enhancing gene expression” is used herein, this refers to an increase in mRNA and/or protein expression which is observed when a particular gene is expressed in a particular expression system from a DNA construct in which a terminator sequence is found downstream of the gene, as compared to expression of the same gene in the same expression system from a DNA construct which is identical to the first DNA construct except that it does not contain a terminator sequence downstream of the gene. Although the level of enhancement is likely to vary between genes, preferably, according to the invention expression of the gene of interest is enhanced from about 10-fold to about 60-fold. Optimum expression for a gene may be achieved using the strong human β-globin poly(A) signal in conjunction with the human β-globin terminator as set out in any of SEQ ID NOS:1, 2, 3 or 4.

Notably, the method of the invention provides an increase in mRNA production and a further increase in protein production. In other words, the use of a terminator element in accordance with the invention results in an increase in the efficiency of transcription termination and so higher levels of mRNA and also results in an increase in the efficiency of translation and so higher levels of protein, as compared to the use of no terminator element.

Advantageously, the amount of nuclear mRNA produced according to the invention is at least 2- to 3-fold greater than the amount produced by a method which is identical except that the DNA molecule does not contain a terminator element. For example, the amount of nuclear mRNA produced according to the invention may be from 2-fold to 20-fold greater, or from 4-fold to 12-fold greater, than previous methods.

Advantageously, the amount of cytoplasmic mRNA produced according to the invention is at least 3-fold greater than the amount produced by a method which is identical except that the DNA molecule does not contain a terminator element. For example, the amount of cytoplasmic mRNA produced according to the invention may be from 3-fold to 40-fold greater, or from 4-fold to 20-fold greater, than previous methods.

Advantageously, the amount of protein produced according to the invention is at least 3-fold greater, preferably at least 10-fold greater, than the amount produced by a method which is identical except that the DNA molecule does not contain a terminator element. For example, the amount of protein produced according to the invention may be from 3-fold to 60-fold greater, preferably from 10-fold to 40-fold greater, than previous methods.

A hallmark of major relevance of terminator elements which encode a section of RNA that is cut co-transcriptionally is their ability to enhance the translation of the resulting mRNA. This may be determined by quantitating mRNA levels, from situations plus and minus the candidate terminator sequence, using RT-PCR (or other such techniques known to the skilled person). Following this, a western blot should be performed to detect the target protein produced from the two situations (plus and minus the candidate CoTC terminator). For the western blot, one must take account of any differences in the mRNA level and adjust the protein input to represent equal levels of mRNA. For instance, if there is twice as much mRNA in one situation than the other then one must add half the amount of protein for the western blot. Even so, more protein is expected to be seen in the presence of a terminator element which encodes a section of RNA that is cut co-transcriptionally, due to the enhancement of translation.

The present invention may be used to enhance the expression of genes contained in an expression vector, such as a plasmid, in vitro. Because increased protein production can be achieved by simply inserting a terminator sequence beyond the gene of interest, this technique is an incredibly cheap and easy technology to implement and requires nothing more than cloning techniques. Further, because the enhancing terminator sequence is positioned downstream of the gene of interest, no alterations in the coding portion of the gene are required.

The invention may also be used to increase the expression of genes integrated into chromosomal locations in cells. For example, a terminator sequence could be integrated downstream of a gene in its chromosomal context using homologous recombination.

The invention may also have an application in gene therapy of a patient where it is desired to increase the expression of a particular protein which is not produced naturally in sufficient levels or in a functional form by the patient. An example of this approach is gene therapy treatment of cystic fibrosis where DNA constructs expressing normal copies of the Cystic Fibrosis Transmembrane Conductance Receptor (CFTR) gene are introduced into patients. This technology could also be useful in gene therapy of a patient where it is desired to increase the expression of a particular protein which is not produced naturally in the patient. Examples of this approach are:

(1) The expression of cytotoxic proteins expressed from genes contained on DNA vectors or constructs directed or introduced into cancerous tumours or tissues for the destruction of said cancerous tumour or tissue.

(2) The expression of proteins expressed from so called ‘suicide genes’ (apoptosis inducing genes) contained on DNA vectors or constructs directed or introduced into cancerous tumours or tissues for the destruction of said cancerous tumour or tissue.

### EXAMPLES

The following examples are illustrative of the products and methods of making the same falling within the scope of the present invention. They are not to be considered in any way limitative of the invention. Changes and modifications can be made with respect to the invention. That is, the skilled person will recognise many possible variations in these examples.

### Example 1

### β-Globin Terminator Sequences Enhance mRNA and Protein Levels of the β-Globin Gene

Pol II termination was studied using the human β-globin gene expressed from transfected plasmids. This process requires a pA signal and downstream terminator element¹. β-globin terminator transcripts are co-transcriptionally cleaved, which presents an uncapped substrate to 5′→3′ exonucleases. Degradation of the trailing transcript precedes termination, after which 3′ end processing takes place^{2, 3}.

Potential roles of Pol II termination in β-globin gene expression were examined. To do this, two plasmids were used: one containing the β-globin gene and its terminator sequence (called βTERM) (SEQ ID NO:1) and another (called βΔTERM), from which the terminator was removed (FIG. 1A). The absence of the terminator reduces termination efficiency by ˜10 fold¹. HeLa cells were transfected with βTERM or βΔTERM along with a co-transfection control plasmid encoding the adenovirus VA gene. Nuclear and cytoplasmic RNA was isolated and gene expression was quantitated by analysing the level of β-globin mRNA, which was detected using real-time RT-PCR. RNA was reverse transcribed with oligo dT and the resulting cDNA was PCR amplified with primers e2 and e3 (FIG. 1A). The presence of the terminator element (βTERM) enhanced nuclear and cytoplasmic β-globin mRNA by 3-4 fold, an effect that we also observed using northern blotting as an alternative assay (lower data panel). In contrast, pre-mRNA levels were similar in βTERM and βΔTERM samples.

The effect of termination on the levels of β-globin protein was then analysed. HeLa cells were transfected with βTERM or βΔTERM as well as a plasmid expressing the HS5 protein, which controls for transfection efficiency. Following this, β-globin and HS5 proteins were detected by western blotting (FIG. 1B). Similar levels of HS5 protein were detected in each case, demonstrating equal transfection efficiency.

However, ten times more β-globin protein was detected in the βTERM protein sample as compared to the βΔTERM sample. These mRNA and protein analyses indicate that termination enhances both mRNA and protein levels.

One consequence of inefficient Pol II termination is the interference of initiation on downstream promoters⁴. On βΔTERM, Pol II does not terminate efficiently and so may interfere with new rounds of initiation as it re-transcribes the promoter sequence. This may provide a trivial explanation for the reduced mRNA levels. In order to quantitate the level of interference, hybrid selection nuclear run on (NRO) analysis was performed on HeLa cells transfected with βΔTERM and VA. Nascent transcripts were radio-labelled and hybridised to a biotinylated probe complementary to the U3 region of the HIV promoter. This region lies adjacent to the promoter (P) region but is only transcribed by Pol II that does not terminate. Selection of the RNA hybrids with streptavidin-coated magnetic beads purifies transcripts continuous with the U3 region, including P transcripts that result from Pol II re-transcribing the promoter. However, P transcripts deriving from new rounds of initiation are not selected (see diagram, FIG. 1C). Selected transcripts (S) and those that escaped selection (NS) were hybridised to separate filters containing anti-sense M13 DNA probes. Most of the U3 signal was in the selected fraction, which demonstrates that the selection was efficient. Even so, the majority of the P signal was not selected, suggesting that it derives from new rounds of initiation and that there is little promoter interference. The experiment was repeated on βTERM transfected HeLa cells and no transcripts were selected because termination prevents transcription of the U3 sequence. More importantly, quantitation and comparison of the P signal in the NS βTERM and βΔTERM fractions revealed that the efficiency of initiation is only reduced to 65% in the latter case. Since this effect is much smaller than the change in protein and mRNA levels seen above, it was concluded that the β-globin terminator element is required for optimal gene expression.

To control for degradation being responsible for the reduced P signal in FIG. 1C, the hybrid selection NRO experiment was repeated on βΔTERM but the position of the selection probe was altered. This time, transcripts upstream of U3 were selected (see FIG. 1D, left diagram). If RNA degradation were responsible for the reduced P signal then few U3 transcripts will be selectable using this upstream probe. However, we were still able to select as many U3 transcripts with the upstream probe as were selected with a probe complementary to U3 itself. Less than 40% of P transcripts were co-selected consistent with initiation only being reduced to around 67% (seen in FIG. 1C). These data indicate that RNA degradation does not prevent the selection of P transcripts.

### Example 2

### Other Terminator Sequences Also Enhance mRNA and Protein Levels of the β-Globin Gene

The effect of three more terminator elements on β-globin gene expression was analysed: one from the mouse serum albumin (MSA) gene⁵ (SEQ ID NO:5), the engineered MAZ4 sequence (SEQ ID NO:6) and the reverse MAZ4 sequence (ZAM4)⁶ (SEQ ID NO:7). Three new plasmids (called βalbTERM, βMAZ4 and βZAM4) were created by inserting either of these elements in place of the β-globin terminator. HeLa cells were transfected with βΔTERM, βalbTERM, βMAZ4 or βZAM4 as well as the VA plasmid. Levels of β-globin mRNA in the nucleus and cytoplasm were then confirmed using the same real-time RT-PCR procedure described in FIG. 1A (FIG. 2A). The presence of any of the three terminator elements resulted in a 3-5 fold stimulation of mRNA levels as compared to βΔTERM (see graph).

We then compared the level of β-globin protein expression from βΔTERM with that from βalbTERM (FIG. 2B), βMAZ4 (FIG. 2C) and βZAM4 (FIG. 2D). Again, the presence of any of the terminator elements resulted in higher levels of β-globin protein as compared to βΔTERM (7 fold increase in protein levels for βalbTERM and 3 fold increase in protein levels for both βMAZ4 and βZAM4). These data provide very strong evidence that an enhancement of gene expression is a general consequence of transcriptional termination.

### Example 3

### Termination Efficiency does not Correlate with Pa Signal Strength

The other cis-acting sequence that is required for termination is the pA signal⁷. It is generally thought that the rate of processing at the pA site determines the efficiency of both gene expression and termination⁸. This relationship was explored in the context of the findings discussed above that termination enhances gene expression. To do so, the effects of the same β-globin terminator element were tested in the presence of pA signals that are processed less efficiently than the β-globin pA signal. The β-globin pA signal in βTERM and βΔTERM was replaced with either the MSA or the human PMScl100 pA signal, to form ATERM, AΔTERM, PMTERM and PMΔTERM. The MSA pA signal is inefficient and the PMScl100 pA signal contains an ATTAAA sequence instead of the AATAAA consensus hexamer, which weakens its processing activity.

Transcriptional termination on ATERM, AΔTERM, PMTERM and PMΔTERM was first analysed using NRO analysis (FIG. 3A). As expected, termination is inefficient on AΔTERM (panel 1) and PMΔTERM (panel 2). This is shown by the high signals over probes A and U3, which detect transcripts from Pol II that fails to terminate. In contrast, termination was close to 100% efficient on both ATERM (panel 3) and PMTERM (panel 4) as shown by the low A and U3 signals. In fact, transcriptional termination is as efficient on ATERM and PMTERM as it is on βTERM, despite the inefficiency of the pA signals used (see graph). Thus, termination efficiency does not correlate with pA signal strength in this system.

Then the real-time RT-PCR strategy outlined in FIG. 1A was used to analyse β-globin mRNA levels in the nucleus and cytoplasm of HeLa cells transfected with ATERM, AΔTERM, PMTERM or PMΔTERM (FIG. 3B). 5-fold more nuclear and 8 fold more cytoplasmic β-globin mRNA was observed in ATERM samples as compared to AΔTERM samples. Similarly nuclear and cytoplasmic β-globin mRNA levels were respectively 10-15 fold higher in PMTERM samples as compared to PMΔTERM samples.

We finally analysed β-globin protein levels in cells transfected with AΔTERM, PMΔTERM, ATERM or PMTERM (FIG. 3C). We observed feint bands of the predicted size for β-globin in the AΔTERM and PMΔTERM samples (lanes 1 and 3). However, strong bands of much greater intensity were observed in the ATERM (25-fold more protein) and PMTERM samples (40-fold more protein) (lanes 2 and 4). Again HS5 levels were equal. These RT-PCR and western blotting data show that gene expression is enhanced even more dramatically by transcriptional termination when 3′ end processing is inefficient. In this situation, transcriptional termination is more influential than the pA signal strength in determining gene expression levels.

### Example 4

### Gene Expression Levels Correlates with Termination Efficiency and not pA Signal Strength

The weak MSA was then compared with the stronger β-globin pA signals in terms of the effect of termination on gene expression. HeLa cells were transfected with βΔTERM, βTERM, AΔTERM or ATERM and β-globin mRNA was detected in the nuclear and cytoplasmic RNA fractions as in FIG. 1A (FIG. 4A). The levels of nuclear β-globin mRNA were similar in βΔTERM and AΔTERM samples. However, 2-3 fold less mRNA was present in the cytoplasm of AΔTERM samples consistent with the MSA pA signal being less efficient in gene expression than the β-globin pA signal. The nuclear level of β-globin mRNA was also similar in βTERM and ATERM nuclear samples but was 4-5 fold greater than with inefficient termination. Interestingly, the presence of the β-globin terminator resulted in near equal levels of cytoplasmic mRNA irrespective of the pA signal used (compare βTERM and ATERM samples).

We sought to confirm the above results and examined β-globin protein levels by western blot analysis of HeLa cells transfected with βΔTERM, AΔTERM, βTERM or ATERM (FIG. 4B). Consistent with the cytoplasmic β-globin mRNA levels, more β-globin protein was observed in the βΔTERM sample (lane 1, relative level 1) as compared to the AΔTERM sample (lane 2, relative level 0.29) and similar, but much higher, amounts for βTERM (lane 3, relative level 10) and ATERM (lane 4, relative level 6.7). These RT-PCR and western blot data show that gene expression levels correlates with termination efficiency and not pA signal strength. In effect, the inefficiency of the MSA pA signal is negated by the efficiency of Pol II termination.

Finally, the nuclear location of β-globin mRNA was analysed using a technique that allows the separation of chromatin associated transcripts from those released into the nucleoplasm⁹. In brief, transfected cell nuclei were treated with urea and detergent followed by centrifugation, which results in the separation of chromatin-associated (present in the pellet) and released RNA (in the supernatant). Nuclei were isolated from HeLa cells transfected with βΔTERM, AΔTERM, βTERM or ATERM and β-globin mRNA was detected from the pellet and released fractions using the RT-PCR procedure described in FIG. 1A (FIG. 4C). For βΔTERM, just over 50% of the mRNA was in the pellet fraction, with slightly more (64%) in the case of AΔTERM. Interestingly, there is a close correlation between the level of released mRNA and the levels of cytoplasmic mRNA. Thus, 75% the mRNA from βTERM and ATERM was in the released fraction, which correlates with the enhanced nuclear and cytoplasmic accumulation of β-globin mRNA from these constructs. This suggests that termination releases mRNA from its site of synthesis and so reduces its susceptibility to nuclear degradation. Indeed, depletion of the PMScl100 subunit of the nuclear exosome enhances nuclear and cytoplasmic levels of β-globin mRNA expressed from AΔTERM (FIG. 4D). This is consistent with transcription levels being similar in the absence of termination, yet mRNA levels are not (FIG. 1C and data not shown).

### Example 5

### β-Globin Terminator Sequences Enhance mRNA and Protein Levels of the Erythropoietin (EPO) Gene

To further generalise these observations, the effects of termination on another gene were analysed. The human erythropoietin (EPO) gene was selected for several reasons. It does not possess a recognisable pA signal, which instead consists of an AAGAAC hexamer¹⁰. Such a signal would not normally function and so would provide a stern test of the effect of termination on gene expression. Second, EPO is a valuable commercial protein and enhancement of its expression would be of significant general interest. The coding sequence of the EPO gene is shown in FIG. 13 and SEQ ID NO:13.

EPO was cloned into βΔTERM and βTERM in place of the human β-globin gene to create EΔTERM and ETERM respectively. These constructs and VA were transfected into HeLa cells and termination efficiency was analysed using a previously described RT-PCR assay that recapitulates termination as seen by NRO¹¹. Nuclear RNA was isolated and reverse transcribed with primer RTr. Following this cDNA was real-time PCR amplified using primers RTr and RTf in order to detect RNA beyond the terminator region (FIG. 12A). As expected, ˜8 fold less read-through RNA was observed for ETERM as compared to EΔTERM, showing that the addition of the terminator region promotes Pol II termination.

EPO mRNA levels in the nucleus and cytoplasm of HeLa cells transfected with EΔTERM or ETERM in addition to VA were next analysed. RNA was reverse transcribed with oligo-dT and then cDNA was real-time PCR amplified using primers EP5′ and EP3′ to detect EPO mRNA (FIG. 12B). Strikingly, much higher levels of nuclear (8 fold) and cytoplasmic (15 fold) EPO mRNA were observed in the ETERM sample as compared to EΔTERM. 3′ RACE analysis confirmed that these mRNAs are processed at the EPO pA signal. This result confirms the observations that relative gene expression enhancement is greater for weak pA signals than for strong poly(A) signals.

The level of EPO protein expression in HeLa cells transfected with EΔTERM or ETERM as well as the HS5 control plasmid was finally analysed. Since EPO is a secreted protein, we examined the culture media for its presence using western blotting. A feint band of the expected size was detected in the EΔTERM sample, whilst a much stronger band was detected in the ETERM sample (FIG. 12C, lower panel). The appearance of a smear most likely results from differential post-translational modification of EPO within the cell, which is well documented. In contrast, the levels of HS5 protein were equal in each case, which shows that equal amounts of cellular protein were loaded into each sample (FIG. 12C, upper panel). These data show that Pol II termination greatly enhances EPO mRNA and protein expression. A mechanism for how termination enhances gene expression is proposed in FIG. 12D.

### Example 6

### Termination Enhances Gene Expression Independent of the Promoter

We next investigated whether termination enhances gene expression in the context of a different promoter with distinct properties to the HIV promoter. The CMV promoter was chosen as it supports high levels of transcription but induces relatively slow elongation¹⁷. This contrasts with the HIV promoter, activated by Tat, which promotes highly processive Pol II elongation^{19, 20}. We replaced the HIV promoter in ETERM and EΔTERM with the CMV promoter, to form CETERM and CEΔTERM respectively. We analysed termination efficiency on these constructs using RT-PCR to quantitate read-through RNA (FIG. 14A). We observed significantly less read-through RNA from CETERM as compared to CEΔTERM, indicating a difference in termination efficiency. These data suggest that a terminator is still required to terminate transcription of the EPO gene.

If termination enhances gene expression, the above results predict that gene expression should be greater for CETERM than for CEΔTERM, since the terminator element improves termination on this construct. This was tested by transfecting HeLa cells with CETERM or CEΔTERM and measuring cytoplasmic mRNA levels (FIG. 14B). 2.5 fold more EPO mRNA was recovered from the CETERM samples as compared to CEΔTERM and a corresponding increase in EPO protein expression was observed (FIG. 14C). These effects on EPO expression are not as great as those observed with the HIV promoter. We conclude that, a terminator is necessary for termination of EPO transcription when transcription is driven by the CMV promoter.

We next tested protein expression in Chinese Hamster Ovary (CHO) cells transfected with CEΔTERM or CETERM (FIG. 14D). As with HeLa cells, the presence of the terminator significantly enhances EPO protein expression, which strongly suggests that the positive effect of termination on gene expression is not cell type specific.

### Example 7

### Mechanism of Increased Gene Expression by Efficient Pol II Termination

We sought to establish why termination enhances gene expression. It is well established that Pol II transcription and pre-mRNA processing are coupled ¹⁶. Since removal of terminator elements inhibits gene expression, we tested whether pre-mRNA splicing efficiency is also reduced. β-globin pre-mRNA splicing was analysed in HeLa cells transfected with βTERM and βΔTERM (FIG. 15A). Nuclear RNA was reverse transcribed with dT to detect cleaved and polyadenylated transcripts, with primer I2r to detect unspliced transcripts or with pAR to detect transcripts not yet cleaved at the pA site. These cDNAs were amplified with primers elf and e2r to detect spliced (S) and unspliced (US) RNA (FIG. 15A). For the dT primed cDNA, only spliced RNA was detected in each case, indicating that the majority of cleaved and polyadenylated transcripts are also spliced. We next amplified the I2r and pAR cDNA with primers elf and e2r to analyse the splicing status of pre-mRNAs. A higher ratio of spliced to unspliced transcripts was recovered from βTERM samples as compared to βΔTERM.

We next analysed splicing of intron 2 using the same RNA samples. Since intron 2 retaining pre-mRNAs are more than 1 kilobase larger than spliced transcripts, primers spanning this region are susceptible to PCR competition. To circumvent this, intron 2 retaining and spliced transcripts were detected from the same pAR primed cDNA, using separate PCR primer pairs: e2f/e3r and e2f/I2r to detect spliced and unspliced transcripts respectively (FIG. 15B). There was a higher ratio of spliced to unspliced transcripts for βTERM as compared to βΔTERM.

A potential criticism of the above result could be that pre-mRNA is degraded in the βTERM sample more efficiently than for βΔTERM. Thus, we repeated our analysis on a further two constructs (βΔTERM1m and βTERM1m), which contain a mutated first intron (FIG. 15C). This mutation prevents splicing but not termination¹⁰ and so allows us to look at differences in the stability of the two pre-mRNAs. Only unspliced transcripts were observed in the analysis and the abundance of pAR and I2r primed cDNAs was unchanged showing that these pre-mRNAs do not have significantly different stabilities.

### Example 8

### The Effect of Termination on Pre-mRNA Splicing is Post-Transcriptional

The enhanced splicing as a result of termination suggests a post-transcriptional effect. We therefore analysed co-transcriptional splicing on βTERM and βΔTERM using a modified NRO protocol to incorporate bromo-labelled UTP (brU) into nascent RNAs, which were purified using a brU-specific antibody¹⁸. We purified brU-labelled RNA from HeLa cells transfected with βTERM or βΔTERM and examined the levels of transcripts containing intron 1 and intron 2 (FIG. 15D). Co-transcriptional splicing is expected to reduce the level of intron containing RNAs that are recovered. cDNA was synthesised with primers e2r or e3r and PCR amplification was with the elf/I1r or e2f/I2r primer pairs to detect intron 1 and 2 respectively. After subtracting the background, obtained from minus antibody controls, we observed little difference in the levels of intron 1 and intron 2 between the βTERM and βΔTERM samples. These data reveal little difference in the co-transcriptional splicing of βTERM and βΔTERM transcripts. The difference in the levels of spliced transcripts observed in total nuclear βTERM and βΔTERM samples is therefore likely to reflect some post-transcriptional splicing as a result of termination.

### Example 9

### The Exosome Degrades Some Transcripts when Termination is Inefficient

We next asked what degrades the transcripts when termination is inefficient. To this end we depleted the nuclear exosome subunit, PMScl100, using RNA interference (RNAi). Western blot analysis of PMScl100 protein in cells that had been mock treated or transfected with PMScl100 specific siRNAs showed that levels were depleted by 2 to 3 fold (FIG. 16A). Equal levels of actin were observed showing that loading was equivalent. These data were substantiated by quantitative RT-PCR analysis of PMScl100 mRNA, which was reduced to 38%. We observed a similar effect with a further two PMscl100 specific short hairpin RNAs (data not shown), which also resulted in similar phenotypes to those described below.

The effect of this depletion was tested in situations where termination and splicing are inefficient and for strong and weak pA signals. Mock and PMScl100 depleted cells were transfected with βΔTERM or AΔTERM and levels of cytoplasmic β-globin mRNA were analysed by RT-PCR (FIG. 16B). We observed increased levels of cytoplasmic mRNA in PMScl100 depleted cells as compared to mock treated cells, identifying PMScl100 as part of the mechanism that suppresses gene expression when termination is inefficient. Interestingly, the effect of exosome depletion was greater for AΔTERM than for βΔTERM. This is in line with our finding that termination enhances gene expression to a greater degree for weaker pA signals. PMScl100 depletion has little effect on βTERM mRNA levels ²¹, which is consistent with the termination process reducing the susceptibility of transcripts to degradation. Depletion of the cytoplasmic exonuclease, Xrn1, had little effect on mRNA levels (data not shown), confirming a nuclear surveillance process.

We next determined the timing of degradation in relation to 3′ end processing. Mock and PMScl100 depleted cells were transfected with AΔTERM and RNA samples were reverse transcribed with pAR (to detect uncleaved) or dT (to detect cleaved and polyadenylated RNAs). Subsequent PCR was with the e3f and e3r primer pair (FIG. 16C). As before, PMScl100 depletion substantially increased the level of RNA cleaved at the pA site (dT primed). However, there was much less of an effect on uncleaved transcripts. These data show that PMScl100 targets AΔTERM transcripts for degradation after cleavage at the pA site. Presumably, the exosome requires free RNA termini to degrade a transcript. For AΔTERM, and other cases where there is no terminator transcript cleavage, this is primarily provided by pA site cleavage. Where terminator transcripts are cleaved, we have shown this to provide additional targets for the exosome ²¹.

### Example 10

### Analysis of mRNA Levels from Linear Templates

ATERM and AΔTERM were linearised (FIG. 17, top two diagrams) by restriction digestion upstream of the promoter (refer to FIG. 3 for description of these plasmids). These constructs, along with the VA control plasmid, were transfected into HeLa cells and nuclear levels of β-globin mRNA were analysed by real-time RT-PCR. Lower diagram shows the primers used for reverse transcription (dT) and PCR (e2f/e3r). We observed ˜2.5 fold higher levels in ATERM samples as compared to AΔTERM samples. In FIG. 3B, an experiment on the same circular templates revealed a 3.8 fold difference in nuclear mRNA levels. The figure of 2.5 fold is ˜65% of this value, which is in good agreement with the reduction in initiation to 62% as determined by hybrid selection NRO (FIG. 18). We therefore conclude that transcription interference effects do not account for the increase in gene expression that is associated with termination. Quantitation shows relative mRNA levels where the AΔTERM value is 1. No pre-mRNA transcripts were detected beyond the linearisation site, showing that the templates remain linear in vivo (data not shown).

Transcriptional interference was quantitated using the assay described in FIG. 1C. Nomenclature is also the same. Analysis was performed on βMAZ4, AΔTERM, PMΔTERM and EΔTERM. For βMAZ4 transcriptional interference was minimal because termination is efficient. In the other cases, initiation was reduced to between 60 and 70%, which is not sufficient to account for the large reduction in protein and mRNA levels observed in FIG. 3. Note, that the addition of the terminator does not affect active Pol II density (FIG. 1C).

### Example 11

### CoTC Terminator Dependent Gene Expression Enhancement in Stably Integrated Genes

To test the possibility that the CoTC transcription termination element enhances gene expression from stably transfected genes in mammalian cell lines we stably integrated βΔTERM and a variant of βΔTERM, labelled βCoTC, which had an insertion of a 370 bp fragment of the β-globin CoTC terminator at a position 200 bp downstream of the β-globin poly(A) site (see FIG. 22A). Both constructs incorporate an HIV promoter which is inducible by addition of the transcriptional activator factor Tat. Pools of stably transfected cells were selected (see Materials and Methods) then transfected with the trans-activating factor Tat to induce gene transcription. Reverse transcription PCR (RT-PCR) analysis was then carried out to detect β-globin messenger RNA (mRNA) from induced and non-induced cells incorporating the βΔTERM and βCoTC genes. The results of this analysis are shown in FIG. 22B.

In FIG. 22B β-globin and EF1A denote the position of RT-PCR products from the integrated β-globin and endogenous EF1A genes respectively. RT-PCR products of the EF1A gene serve as a loading control. The diagrams above the data panel indicate cells that have integrated the βCoTC construct (lanes 1 and 2) and cells that have integrated the βΔTERM construct (lanes 3 and 4). For cells that have integrated the βCoTC construct there is a low level of β-globin RT-PCR product in the absence of Tat induced transcriptional activation (lane 1). (This low level of transcription probably derives from ‘readthrough’ transcription from genes adjacent to the site of βΔTERM and βCoTC integration. This proposition is borne out by the control experiment (lane 5) where RT-PCR analysis of control cells, lacking stable integrants, shows there is no background β-globin mRNA, indicating that all β-globin PCR products derive from the integrated genes). Upon addition of Tat there is a significant increase in the abundance of β-globin mRNA from cells containing the βCoTC gene (lane 2), reflecting the high level of Tat induced transcription from the HIV promoter.

However for cells that have integrated βΔTERM there is a low level of β-globin mRNA in the absence of Tat induced transcriptional activation (lane 3), which remains unchanged even upon addition of Tat (lane 4). This result indicates that the CoTC Termination element is required for high level gene expression.

The most important part of this experiment is the comparison of gene expression levels from transcriptionally induced integrated genes that do or do not contain the CoTC Terminator element. Comparison of βCoTC (lane 2) and βΔTERM (lane 4) shows that the CoTC Terminator element significantly enhances stably integrated β-globin gene expression. Thus it appears that CoTC Terminator element dependent gene expression enhancement, that we have examined in detail in transient transfection analyses, extends to genes that are located in a chromosomal context.

### Example 12

### CoTC Terminator Dependent Protein Expression Enhancement in Plants

To test the possibility that the CoTC transcription termination element enhances protein expression in plants we made an expression plasmid containing a YFP (Yellow Flourescent Protein)/RAB reporter gene, positioned upstream of the octopine synthase gene poly(A) signal, labelled pYFP/RAB (see FIG. 23A, upper diagram). A variant of this construct, labelled pYFP/RAB+CoTC, was also made by insertion of a 370 bp fragment of the β-globin CoTC Terminator approximately 300 bp downstream of the poly(A) signal (FIG. 23A, lower diagram).

Agrobacterium were transformed with pYFP/RAB and pYFP/RAB+CoTC. The resulting Agrobacterium clones were then infiltrated onto different tobacco plant leaves. YFP fluorescence was quantified from 12 confocal images of each leaf sector and background was subtracted. The data from two such experiments were combined and are displayed in the graph shown in FIG. 23B. It is apparent that YFP expression levels are higher in leaf cells transfected with the Agrobacterium pYFP/RAB+CoTC clone. The increased abundance of the YFP/RAB fusion protein is due to the enhancement effect of the CoTC terminator element noted in mammalian cells

Materials and Methods

Nuclear run on and hybrid selection nuclear run on The protocols for NRO and hs NRO have previously been described in detail^{10, 11}. The M13 probes: P¹⁰, B3 and B4¹¹, A³, U3 and VA¹⁰ have also been described. The template from which the U3 selection probe was made was constructed by inserting an AvaI/PvuII restriction fragment from βTERM into pGEM4 and the upstream U3 probe was made by PCR amplification of βΔTERM with primers U35′ and U3T7. These clones were transcribed by SP6 and T7 polymerase¹⁰. The brUNRO protocol was performed as described¹⁸.

Real-Time RT-PCR

cDNA was made using SuperScript III (Invitrogen) and 1 ul of the 20 ul reaction was subsequently analysed by real-time PCR (10 pmol of each oligo, 1 ul of cDNA, 7.5 ul of SYBR green mix (Qiagen) and water to a final volume of 15 ul) or semi-quantitative PCR (Taq polymerase (Bioline) (1 ul 10 mM dNTPs, 10 pmol each primer, 1.5 mM magnesium chloride, 1× manufacturers buffer). Graphs show average and standard deviation of signals obtained from between 3 and 12 biological repeats. Experiments were quantitated after subtraction of values obtained from minus RT samples.

Western Blotting

Western blotting was performed as described in¹². 50% of lysate from a confluent 5 cm dish of HeLa cells was used for analysis. For secreted EPO, 10-100 ul of culture media was used. Membranes were probed with anti-human β-globin (Santa Cruz) at 1:1000, anti-PMScl100 (Abcam) at 1:1000, anti-actin (Sigma) at 1:1000 or anti HA (Santa Cruz) at 1:1000. Secondary antibodies were anti-mouse (Sigma) at 1:2000 or anti-rabbit (Sigma) at 1:2000. Signals were detected with an ECL kit (GE healthcare) and quantitated using image quant software. EPO protein was detected using the EPO (B-4) antibody (Santa Cruz) at a 1:500 dilution. To detect EPO, 10-100 ul of culture media were analysed for the secreted protein.

Northern Blotting

The protocol used by the Narry Kim lab for detecting small RNA was used (http://www.narrykim.org/Northern_blot_analysis_for_microRNA.pdf). RNA samples were RNase H cleaved using primer 4.5 and dT. RNA was fractionated on a 6% gel and products detected using 5′ 32P-labelled e3r primer.

Transfections

Transient Transfection

Semi-confluent HeLa cells, in 5 or 10 cm plates, were transfected with 1-5 ug of reporter plasmid, 1-2 ug of VA plasmid and 1.5 ug of Tat plasmid. Lipofectamine 2000 (Invitrogen) was used in accordance with the manufacturer's guidelines. RNA or protein was isolated 12-20 hours post transfection.

Stable Transfection

HeLa cells were transfected as for transient transfection with βΔTERM or βCoTC along with 1-2 ug of pCl-neo (Promega Corp.) a plasmid encoding the neo gene which confers G418 resistance on transfected cells. Pools of stable integrants were then created and maintained by continuous antibiotic selection, according to Sambrook et al.²⁴.

Transient Expression in Plants

Agrobacterium mediated transformation and YFP fluorescence measurements were carried out as described²⁵.

RNA Isolation

The procedure for isolating nuclear and cytoplasmic RNA has been described¹² as has our protocol for separating nuclear RNA into chromatin-associated and released fraction¹³.

Nuclear RNA Fractionation

This protocol was originally described in⁹. However, our protocol used it with modifications described in¹³.

RNAi Interference

RNAi interference of PMScl100 is described in ²¹.

Quantitation

Quantitation is shown as an average of at least 3 independent experiments. Errors are standard deviations from the mean. Error margins are provided where average effects were less than 10 fold.

Plasmid Constructs

The Tat²², VA¹⁰, βTERM and βΔTERM (previously called βΔ5-7 and βΔ5-10)¹; βMAZ4 (previously called pMAZ4), βZAM4 (previously called pZAM4), βmMAZ4 (previously called pmMAZ4) ⁶; AΔTERM (previously called AΔ5-10) and βalbTERM (previously called βalb) plasmids⁵ have been described previously. ATERM was made by inserting a TERM5′/TERM3′ PCR product into a vector prepared by PCR amplification of βΔ5-10ApA using the APR/RTf primers. PMΔTERM and PMTERM were made by inserting a PCR product, generated by PMF/PMR amplification of HeLa cell DNA, into vectors prepared by F/e3 PCR amplification of βΔTERM or βTERM respectively. AMAZ4 was made by inserting an APF/APR PCR product into a vector generated by PCR amplification of βMAZ4 with the F/e3r primer pair.

The EPO gene was amplified from HeLa cell genomic DNA, using primers E5′/E3′. EΔTERM was made by inserting EPO into a vector prepared by PCR amplification of βΔTERM with primers TAR3′ and RTf. ETERM was made by inserting EPO into a vector prepared by PCR amplification of βTERM using primers TAR3′ and TERM5′. The RBM21 expression plasmid was a kind gift from Chris Norbury. RBM21 is a member of the recently discovered family of non-canonical poly(A) polymerases. The βpA and MSA competition clones are described elsewhere⁵. The PMScl100pA competition clone was made by inserting a PMF/PMR PCR product into a vector made by PCR amplification of the βpA competition clone using primers SPAf and e3. For the CMV promoter constructs, the HIV promoter was removed by an AvaI/AflII digest and the CMV promoter, obtained by BglII/HindIII digest of pcDNA3.1 (Invitrogen), was inserted.

βCoTC, was made by insertion of a 370 bp fragment of the β-globin CoTC terminator (a PCR product made by PCR amplification of βTERM with primers TERM5′ and COTC3′) into a position 200 bp downstream of the βΔTERM poly(A) site.

pYFP-RAB was made by PCR amplification, using a proof reading polymerase, of a YFP-RAB fusion gene from a plasmid labelled pBIN-YFPAZa (gift from I. Moore) using primers YFPf and RABr. 3′ A-overhangs were then added to the resulting YFP-RAB fusion gene PCR product, using Taq polymerase, before cloning into pCR8®/GW/TOPO (Invitrogen) forming pCR8®/GW/TOPO/YFP-RAB. The YFP-RAB fusion gene insert was then transferred from pCR8®/GW/TOPO/YFP-RAB into an expression vector labelled pOpIN1 (gift from I. Moore), upstream of the octopine synthase poly(A) site, using Gateway cloning technology (Invitrogen) to form pYFP-RAB. Construct pYFP-RAB+CoTC was made by addition of a 370 bp fragment of the β-globin CoTC terminator (a PCR product made by amplification of βTERM with primers TERM5′ and COTC3′) into a unique Not1 restriction site positioned ˜300 bp downstream of the octopine synthase poly(A) site in pYFP-RAB.

Primers (5′→3′)

| | |
|---|---|
| e2f: | TGGCCTGGCTCACCTGGACAACC |
| e3r: | ATCCAGATGCTCAAGGCCC |
| RTFVF: | CAGGAAACTATTACTCAAAGGGTA |
| VR/pAR: | CTTGAATCCTTTTCTGAGGGATG |
| RTr: | AGAAAATACCGCATCAGGCGCCAT |
| TAR3′ | GAGCTTTATTGAGGCTTAAGCAG |
| TERM5′ | GCATAGTGTTACCATCAACCA |
| TERM3′ | TTTCCTGATTCTCCCACCCCC |
| PMF | GCTTCAGGTACAACTGGCCAC |
| PMR | GGAGCACACTCACCTGCCCAC |
| EP5′/EPf′ | AAGCTGTGACTTCTCCAGGTC |
| EP3′/EPr′ | TGGTTTCAGTTCTTGTCAATG |
| E5′ | ATGGGGGTGCACGGTGAGTAC |
| E3′ | TCAGACAGGCTGTGTGAGACAG |
| APR | AAAGGCAGGGATTCCTCTGAGCC |
| APF | CCCTAAGGAACACAAATTTCTTTA |
| pAF | CCACAAGTATCACTAAGCTCGC |
| R | AACTAGCTCTTCATTTCTTTATG |
| F | CCTTGGGAAAATACACTATATC |
| 4.5 | TTGTGGGCCAGGGCATTAGCCACA |
| SPAf | CCTTGGGAAAATACACTATATC |
| 12r | CTATGACATGAACTTAACCATAG |
| elf | ACTCCTGAGGAGAAGTCTGCC |
| e2r | TTTCTTGCCATGACCCTTCACC |
| Ilr | TCAGTGCCTATCAGAAACCC |
| e3f | CCACAAGTATCACTAAGCTCGC |
| U35′ | TTACGGTTCCTGGCCTTTTGCTGG |
| COTC3′ | CGGCTGCAACATGAATATTAG |
| YFPf | ACCATGGGATCCAGTGAGCAAGG |
| RABr | TCAAGACGATGAGCAACAAGGC |
| U3T7 | TAATACGACTCACTATAGGGAGGTTT |
| | CCTGTGTGAAATTGTTATCCGC |

### REFERENCES

1. Dye, M. J. & Proudfoot, N. J. Multiple transcript cleavage precedes polymerase release in termination by RNA polymerase II. Cell 105, 669-81 (2001).
2. West, S., Proudfoot, N. J. & Dye, M. J. Molecular dissection of mammalian RNA polymerase II transcriptional termination. Mol. Cell 29, 600-10 (2008).
3. West, S., Gromak, N. & Proudfoot, N. J. Human 5′-->3′ exonuclease Xrn2 promotes transcription termination at co-transcriptional cleavage sites. Nature 432, 522-5 (2004).
4. Greger, I. H., Aranda, A. & Proudfoot, N. Balancing transcriptional interference and initiation on the GAL7 promoter of Saccharomyces cerevisiae. Proc. Natl. Acad. Sci. U.S.A. 97, 8415-20 (2000).
5. West, S., Zaret, K. & Proudfoot, N. J. Transcriptional termination sequences in the mouse serum albumin gene. RNA 12, 655-65 (2006).
6. Gromak, N., West, S. & Proudfoot, N. J. Pause sites promote transcriptional termination of mammalian RNA polymerase II. Mol. Cell. Biol. 26, 3986-96 (2006).
7. Whitelaw, E. & Proudfoot, N. Alpha-thalassaemia caused by a poly(A) site mutation reveals that transcriptional termination is linked to 3′ end processing in the human alpha 2 globin gene. EMBO J 5, 2915-22 (1986).
8. Edwalds-Gilbert, G., Prescott, J. & Falck-Pedersen, E. 3′ RNA processing efficiency plays a primary role in generating termination-competent RNA polymerase II elongation complexes. Mol. Cell. Biol. 13, 3472-80 (1993).
9. Wuarin, J. & Schibler, U. Physical isolation of nascent RNA chains transcribed by RNA polymerase II: evidence for cotranscriptional splicing. Mol. Cell. Biol. 14, 7219-25 (1994).
10. Dye, M. J. & Proudfoot, N. J. Terminal exon definition occurs cotranscriptionally and promotes termination of RNA polymerase II. Mol. Cell 3, 371-8 (1999).
11. Ashe, H. L., Monks, J., Wijgerde, M., Fraser, P. & Proudfoot, N. J. Intergenic transcription and transinduction of the human beta-globin locus. Genes Dev. 11, 2494-509 (1997).
12. West, S. & Proudfoot, N. J. Human Pcfl1 enhances degradation of RNA polymerase II-associated nascent RNA and transcriptional termination. Nucleic Acids Res. 36, 905-14 (2008).
13. Dye, M. J., Gromak, N. & Proudfoot, N. J. Exon tethering in transcription by RNA polymerase II. Mol. Cell 21, 849-59 (2006).
14. Tantravahi, J., Alvira, M. and Falck-Pedersen, E. Characterization of the mouse beta^{maj} globin transcription termination region: a spacing sequence is required between the poly(A) signal sequence and multiple downstream termination elements. Mol. Cell. Biol. 13, 578-587 (1993).
15. Plant, K. E., Dye, M. J., Lafaille, C. & Proudfoot, N. J. Strong polyadenylation and weak pausing combine to cause efficient termination of transcription in the human Ggamma-globin gene. Mol. Cell. Biol. 25, 3276-85 (2005).
16. Proudfoot, N. J., Furger, A., and Dye, M. J. (2002). Integrating mRNA processing with transcription. Cell 108, 501-512.
17. Cramer, P., Pesce, C. G., Baralle, F. E., and Kornblihtt, A. R. (1997) Functional association between promoter structure and transcript alternative splicing. Proc. Natl. Acad. Sci. USA 94, 11456-11460.
18. Lin, S., Coutinho-Mansfield, G., Wang, D., Pandit, S., and Fu, X. D. (2008). The splicing factor SC35 has an active role in transcriptional elongation. Nat. Struct. Mol. Biol. 15, 819-826.
19. Nogues, G., Kadener, S., Cramer, P., Bentley, D., and Kornblihtt, A. R. (2002). Transcriptional activators differ in their abilities to control alternative splicing. J. Biol. Chem. 277, 43110-43114.
20. Parada, C. A., and Roeder, R. G. (1996). Enhanced processivity of RNA polymerase II triggered by Tat-induced phosphorylation of its carboxy-terminal domain. Nature 384, 375-378.
21. West, S., Gromak, N., Norbury, C. J., and Proudfoot, N. J. Adenylation and exosome-mediated degradation of cotranscriptionally cleaved pre-messenger RNA in human cells. Mol. Cell 21, 437-443.
22. Adams, S. E., Johnson, I. D., Braddock, M., Kingsman, A. J., Kingsman, S. M., and Edwards, R. M. (1988). Synthesis of a gene for the HIV transactivator protein Tat by a novel single stranded approach involving in vivo gap repair. Nucleic Acids Res. 16, 4287-4298.
23. Samarsky, D. A., Ferbeyre, G., Bertrand, E., Singer, R. H., Cedergren, R., and Fournier, M. J. (1999). A small nucleolar RNA:ribozyme hybrid cleaves a nucleolar RNA target in vivo with near-perfect efficiency. Proc. Natl. Acad. Sci. U.S.A. 96, 6609-6614.
24. Sambrook, J., Fritsch, E. F. and Maniatis, T. (1989) Molecular Cloning—a laboratory manual. 2nd Edition. Cold Spring Harbor Laboratory Press. Cold Spring Harbor. N.Y.
25. Johansen, J. N., Chow, C-M., Moore, I. and Hawes, C. AtRAB-H1^{b} and AtRAB-H1^{c} GTPases, homologues of the yeast Ypt6, target reporter proteins to the Golgi when expressed in Nicotiana tabacum and Arabidopsis Thaliana. J. Exp. Bot. Advance access published May 26, 2009, doi:10.1093/jxb/erp153.

1. A method of enhancing expression of a gene of interest comprising providing an isolated DNA molecule having a sequence which comprises in a 5′ to 3′ direction (i) one or more promoter elements, (ii) the gene of interest, and (iii) a poly-adenylation signal, and (iv) a terminator element, and expressing the gene of interest incorporated into the DNA molecule in an expression system.

2. A method according to claim 1 wherein the terminator element encodes a section of RNA that is cut co-transcriptionally.

3. A method according to claim 1 wherein the terminator element encodes a section of RNA that comprises (i) a CoTC substrate or (ii) a ribozyme.

4. A method according to claim 3 wherein the terminator element encodes a section of RNA that comprises a ribozyme and a pause type terminator sequence.

5. A method according to claim 1 wherein the terminator element comprises at least about 250 nucleotides.

6. A method according to claim 1 wherein the terminator element is AU rich in that the RNA encoded by this element contains at least 60% A and/or U residues.

7. A method according to claim 1 wherein the terminator element comprises one or more terminator elements selected from the sequences of SEQ ID NOS: 1 to 12 and 45, or a fragment or variant thereof, or any combination thereof.

8. A method according to claim 1 wherein the terminator element comprises the human β-globin terminator region as set out in SEQ ID NO:1 or a fragment or variant thereof, or the terminator element comprises one or more of elements 8, 9 and 10 of the human β-globin terminator sequence as set out in SEQ ID NO:2, SEQ ID NO:3 and SEQ ID NO:4, respectively, or a variant thereof.

9. A method according to claim 1 wherein the poly-adenylation signal is selected from a sequence comprising AATAAA, ATTAAA, the MSA poly-adenylation signal, the EPO poly-adenylation signal and the PMScl100 poly-adenylation signal.

10. A method according to claim 1 wherein the expression system is selected from a culture of mammalian cells, insect cells, plant cells, bacterial cells or yeast cells, or a cell-free system.

11. A method according to claim 1 provided that the gene of interest is not the human β-globin gene, the human ε-globin gene, the human β-actin gene, the human gamma A globin gene, the human gamma G globin gene or the mouse β-major globin gene.

12. A method according to claim 1 wherein the terminator sequence is located from 0 to 5000 bp downstream of the poly(A) signal, preferably from 150 to 4000 bp downstream of the poly(A) signal.

13. A method according to claim 1 wherein the gene of interest is erythropoietin.

14. A method according to claim 1 wherein expression of the gene of interest is enhanced at least 10-fold.

15. A method according to claim 1 wherein the amount of nuclear mRNA and/or the amount of cytoplasmic mRNA produced is at least 2-fold greater than the amount produced by a method which is identical except that the DNA molecule does not contain a terminator element.

16. A method according to claim 1 wherein the amount of protein produced is at least 10-fold greater than the amount produced by a method which is identical except that the DNA molecule does not contain a terminator element.

17. An isolated DNA molecule having a sequence which comprises in a 5′ to 3′ direction (i) one or more promoter elements, (ii) a gene of interest, (iii) a poly-adenylation signal, and (iv) a terminator element, provided that the gene of interest is not the human β-globin gene, the human ε-globin gene, the human β-actin gene, the human gamma A globin gene, the human gamma G globin gene or the mouse β-major globin gene.

18. An isolated DNA molecule according to claim 17 wherein the terminator element encodes a section of RNA that is cut co-transcriptionally.

19. An isolated DNA molecule according to claim 17 wherein the terminator element comprises (i) a CoTC substrate or (ii) a ribozyme.

20. An isolated DNA molecule according to claim 17 wherein the terminator element comprises one or more terminator elements selected from the sequences of SEQ ID NOS: 1 to 12 and 45, or a fragment or variant thereof, or any combination thereof.

21. An isolated DNA molecule according to claim 17 wherein the terminator element comprises the human β-globin terminator region as set out in SEQ ID NO:1 or a fragment or variant thereof, or the terminator element comprises one or more of elements 8, 9 and 10 of the human β-globin terminator sequence as set out in SEQ ID NO:2, SEQ ID NO:3 and SEQ ID NO:4, respectively, or a variant thereof.

22. An isolated DNA molecule according to claim 17 wherein the gene of interest is erythropoietin.

23. A process for the production of a polypeptide which comprises expression of the coding sequence incorporated into a DNA molecule according to claim 17.

24. A process according to claim 23 wherein the polypeptide is produced in an expression system selected from a culture of mammalian cells, insect cells, plant cells, bacterial cells or yeast cells, or a cell-free system.

25. An isolated DNA molecule according to claim 17 for use in therapy.

26-27. (canceled)
